SEQUENCE LISTING (75 sequences)

SEQ ID NO: 1 - protein, 10 aa, Homo sapiens
GVYDGREHTV

SEQ ID NO: 2 - protein, 10 aa, Homo sapiens
GLADGRTHTV

SEQ ID NO: 3 - protein, 10 aa, Homo sapiens
GVPDCRIFTV

SEQ ID NO: 4 - protein, 10 aa, Homo sapiens
SVYDAREFSV

SEQ ID NO: 5 - protein, 10 aa, Homo sapiens
GLSDGQWHTV

SEQ ID NO: 6 - protein, 10 aa, Homo sapiens
GVFDNCSHTV

SEQ ID NO: 7 - protein, 10 aa, Homo sapiens
KVSDGHFHTV

SEQ ID NO: 8 - skipped

SEQ ID NO: 9 - protein, 10 aa, Homo sapiens
ALVDQRELYL

SEQ ID NO: 10 - skipped

SEQ ID NO: 11 - protein, 10 aa, Homo sapiens
ALLDYREDGV

SEQ ID NO: 12 - protein, 10 aa, Homo sapiens
CLIDDREMPV

SEQ ID NO: 13 - protein, 10 aa, Homo sapiens
LLDDNRELLV

SEQ ID NO: 14 - protein, 10 aa, Homo sapiens
VLVDDRECPV

SEQ ID NO: 15 - protein, 10 aa, Homo sapiens
ILIDIREFTL

SEQ ID NO: 16 - protein, 10 aa, Homo sapiens
VLIDIREYWM

SEQ ID NO: 17 - protein, 10 aa, Homo sapiens
YSMDIREFQL

SEQ ID NO: 18 - protein, 10 aa, Homo sapiens
YLADEREDLL

SEQ ID NO: 19 - protein, 10 aa, Homo sapiens
ILLDNREYLA

SEQ ID NO: 20 - protein, 10 aa, Homo sapiens
MLWQGRDDHV

SEQ ID NO: 21 - protein, 10 aa, Homo sapiens
IIFDARQNSV

SEQ ID NO: 22 - protein, 10 aa, Homo sapiens
LLFQGRNPGV

SEQ ID NO: 23 - protein, 10 aa, Homo sapiens
FMFEGREIKL

SEQ ID NO: 24 - protein, 10 aa, Homo sapiens
QMFDARNMMA

SEQ ID NO: 25 - protein, 10 aa, Homo sapiens
YMYQARDLAA

SEQ ID NO: 26 - protein, 10 aa, Homo sapiens
YMFEAREFLR

SEQ ID NO: 27 - protein, 10 aa, Homo sapiens
LLMDARDLVL

SEQ ID NO: 28 - protein, 10 aa, Homo sapiens
KMMEGRNSSI

SEQ ID NO: 29 - protein, 9 aa, Homo sapiens
EVDPIGHLY

SEQ ID NO: 30 - protein, 9 aa, Homo sapiens
EVGPIFHLY

SEQ ID NO: 31 - protein, 9 aa, Homo sapiens
EVVRIGHLY

SEQ ID NO: 32 - protein, 9 aa, Homo sapiens
YVDSEGHLY

SEQ ID NO: 33 - protein, 9 aa, Homo sapiens
EVDKIGRGY

SEQ ID NO: 34 - protein, 9 aa, Homo sapiens
EVDQIYHLA

SEQ ID NO: 35 - protein, 9 aa, Homo sapiens
VVDNIDHLY

SEQ ID NO: 36 - protein, 9 aa, Homo sapiens
EVDELVHLY

SEQ ID NO: 37 - protein, 9 aa, Homo sapiens
EVSPDGELY

SEQ ID NO: 38 - protein, 9 aa, Homo sapiens
EDDVIEHLY

SEQ ID NO: 39 - protein, 9 aa, Homo sapiens
EVDPTSHSY

SEQ ID NO: 40 - protein, 9 aa, Homo sapiens
WNIPIGLLY

SEQ ID NO: 41 - protein, 9 aa, Homo sapiens
FVGEIGDLY

SEQ ID NO: 42 - protein, 9 aa, Homo sapiens
ELDPEGSLH

SEQ ID NO: 43 - protein, 9 aa, Homo sapiens
DVNGIRHLY

SEQ ID NO: 44 - protein, 9 aa, Homo sapiens
EVAPAGASY

SEQ ID NO: 45 - protein, 9 aa, Homo sapiens
ELDPIQKLF

SEQ ID NO: 46 - protein, 9 aa, Homo sapiens
ESLPKDHLY

SEQ ID NO: 47 - protein, 9 aa, Homo sapiens
ESDPIVAQY

SEQ ID NO: 48 - protein, 9 aa, Homo sapiens
KVLEHVVRV

SEQ ID NO: 49 - protein, 9 aa, Homo sapiens
KVLEHVVRV

SEQ ID NO: 50 - protein, 9 aa, Homo sapiens
KVMCHVRRV

SEQ ID NO: 51 - protein, 9 aa, Homo sapiens
KVLERVNAV

SEQ ID NO: 52 - protein, 9 aa, Homo sapiens
NVTEYVVRV

SEQ ID NO: 53 - protein, 9 aa, Homo sapiens
FLLETVVRV

SEQ ID NO: 54 - protein, 9 aa, Homo sapiens
KVLEQPVVV

SEQ ID NO: 55 - protein, 9 aa, Homo sapiens
MVLEHPARV

SEQ ID NO: 56 - protein, 9 aa, Homo sapiens
KVLQNVLRV

SEQ ID NO: 57 - protein, 9 aa, Homo sapiens
KVLGIVVGV

SEQ ID NO: 58 - protein, 9 aa, Homo sapiens
KQMEHVQRV

SEQ ID NO: 59 - protein, 9 aa, Homo sapiens
KVLESGVNV

SEQ ID NO: 60 - protein, 9 aa, Homo sapiens
KVLEILHRV

SEQ ID NO: 61 - protein, 9 aa, Homo sapiens
GVLEHIGRV

SEQ ID NO: 62 - protein, 9 aa, Homo sapiens
AVEEHVVSV

SEQ ID NO: 63 - protein, 9 aa, Homo sapiens
KVIECNVRV

SEQ ID NO: 64 - protein, 9 aa, Homo sapiens
VVLEPVERV

SEQ ID NO: 65 - protein, 9 aa, Homo sapiens
KVLECCHRV

SEQ ID NO: 66 - protein, 9 aa, Homo sapiens
KILEDVVGV

SEQ ID NO: 67 - protein, 9 aa, Homo sapiens
KVLEGVVAA

SEQ ID NO: 68 - protein, 9 aa, Homo sapiens
KVLETLVTV

SEQ ID NO: 69 - protein, 9 aa, Homo sapiens
KVLESVFRI

SEQ ID NO: 70 - protein, 9 aa, Homo sapiens
KVLEHVPLL

SEQ ID NO: 71 - protein, 9 aa, Homo sapiens
KVLEEVDFV

SEQ ID NO: 72 - protein, 9 aa, Homo sapiens
EVLENVFRV

SEQ ID NO: 73 - protein, 10 aa, Homo sapiens
GLYDGREHSV

SEQ ID NO: 74 - protein, 10 aa, Homo sapiens
GLYDGMEHLI

SEQ ID NO: 75 - protein, 6 aa, synthetic construct
HHHHHH

(19) United States
(12) Patent Application Publication
(10) Pub. No.: US 2025/0014675 A1
(43) Pub. Date: Jan. 9, 2025

(54) OFF-TARGET PREDICTION METHOD FOR ANTIGEN-RECOGNITION MOLECULES BINDING TO MHC-PEPTIDE TARGETS

(71) Applicant: Regeneron Pharmaceuticals, Inc., Tarrytown, NY (US)

(72) Inventors: Ankur Dhanik, Pleasantville, NY (US); Kunal Kundu, Tuckahoe, NY (US); Wen-yi Lee, New Hyde Park, NY (US); David Suh, Midland Park, NJ (US); Gang Chen, Yorktown Heights, NY (US); Robert Salzler, Ossining, NY (US); Robert Babb, River Edge, NJ (US)

(73) Assignee: Regeneron Pharmaceuticals, Inc., Tarrytown, NY (US)

(21) Appl. No.: 18/749,036

(22) Filed: Jun. 20, 2024

Related U.S. Application Data
(63) Continuation of application No. PCT/US22/82070, filed on Dec. 20, 2022.
(60) Provisional application No. 63/292,205, filed on Dec. 21, 2021.

(51) Int. Cl. G16B 15/30; C12N 15/10; G01N 33/564

(52) U.S. Cl. CPC G16B 15/30; C12N 15/1037; G01N 33/564; G01N 2333/70539

(57) ABSTRACT

Computational systems and methods for predicting amino acid position(s) within a target peptide presented in a complex with a major histocompatibility complex (MHC) molecule (MHC-target peptide complex), the amino acid position(s) being involved in interacting with an antigen-recognition molecule that recognizes said MHC-target peptide complex, are presented herein. Computational systems and methods for estimating a number of off-target peptide(s) for an antigen-recognition molecule that recognizes a target peptide presented in a complex with a major histocompatibility complex (MHC) molecule (MHC-target peptide complex) is presented herein. Computational systems and methods for ranking potential target peptides to mitigate off-target toxicity are presented herein. Such computational systems and methods can streamline development of effective, well tolerated antigen-recognition molecules to treat diseases.

### CROSS-REFERENCE TO RELATED APPLICATIONS

This application is a continuation of International Application Serial No. PCT/US2022/082070 filed on Dec. 20, 2022 and published as WO2023/122621, which claims priority to U.S. Provisional Application Ser. No. 63/292,205 filed on Dec. 21, 2021, the contents of each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 5, 2023, is named 250298_000435_SL.xml and is 64,055 bytes in size.

### FIELD

The present invention relates generally to computational systems and methods for predicting amino acid positions involved in interacting with an antigen-recognition molecule within a target peptide presented in a complex with a major histocompatibility complex (MHC) molecule (MHC-peptide complex), and for predicting off-target peptides for an antigen-recognition molecule that recognizes a target peptide in an MHC-peptide complex.

### BACKGROUND

Antigen-recognition molecules, such as T-cell receptors (TCRs) and antibodies, are capable of identifying antigens, which include agents recognized by the immune system of a host as defined herein. Antigen-recognition molecules can help the immune system neutralize an antigen by binding to an antigenic peptide presented in a complex with a major histocompatibility complex (MHC) molecule (MHC-peptide complex) on the surface of an antigen-presenting cell.

The MHC-peptide complex is presented on the surface of the antigen-presenting cell as a result of a cellular process in which an MHC gene in the antigen cell encodes an MHC molecule; the MHC molecule subsequently binds to the antigenic peptide thereby creating the MHC-peptide complex; and the resulting MHC-peptide complex is positioned on the cell surface so that a portion of the peptide is presented for binding with an antigen-recognition molecule. Each peptide is made up of a short chain of amino acids, and some of the amino acids of a peptide in an MHC-peptide complex are bound to the MHC molecule while at least some of the remaining amino acids are presented as available for binding with an antigen-recognition molecule.

This cellular process is also carried out in cells native to the body. Generally, antigen-recognition molecules are able to distinguish between peptides presented on native cells vs. peptides presented on antigen-presenting cells so that normal cells are not attacked by the immune system. Antigen-recognition molecules are able to bind to a peptide of an MHC-peptide complex on the surface an antigen-presenting cell to help the immune system neutralize the antigen.

Research is underway with a goal of engineering antigen-recognition molecules to target cells that would otherwise not be targeted through the above-described mechanism. For instance, cancer cells are native cells that are not effectively suppressed by the immune system, and research has shown that it can be possible to engineer TCRs, antibodies, and other antigen-recognition molecules to target the cancer-specific MHC-peptide complexes on cancer cells. While targeted treatments with engineered antigen-recognition molecules may be effective to neutralize intended target cells, side effects of the treatment may be severe if engineered antigen-recognition molecules attack off-target native cells in addition to the intended target cells. Side effects are often identified during clinical trials, which can result in patient death, other adverse effects on patients, and expenditure of time and resources in research and development. Accordingly, there is a need in the art for methods and systems that allow for accurate and efficient prediction of off-targets to the target peptide of interest which helps evaluate the risk associated with the target peptide at the target selection step as well as help in screening the most specific antigen-recognition molecules.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes non-transitory computer-readable medium configured to communicate with one or more processor(s) of a computational device. The non-transitory computer-readable medium includes instructions to perform the following steps, which can be performed in various orders and with interleaving steps: a) receive, as an input, a computational representation of a target peptide presented in a MHC-target peptide complex; b) determine binding affinity of the target peptide to the MHC molecule of the MHC-target peptide complex; c) generate sequences of a plurality of mutated peptides each associated with a mutation at a respective amino acid position of the target peptide; d) determine binding affinity of each mutated peptide of the plurality of mutated peptides to the MHC molecule; e) predict the amino acid position(s) involved in interacting with an antigen-recognition molecule that recognizes said MHC-target peptide complex based at least in part on a comparison of the binding affinity for each mutated peptide to the binding affinity of the target peptide; and f) provide, as an output, an indication of the amino acid position(s) likely to be involved in interacting with the antigen-recognition molecule. Other embodiments of this aspect include corresponding computer systems, apparatus, and computer programs recorded on one or more computer storage devices, each configured to perform the actions of the methods.

Implementations may include one or more of the following features. The non-transitory computer-readable medium may include instructions that, when executed by the processor(s), cause the computational device to: predict that a respective position of the plurality of positions is involved in interacting with an antigen-recognition molecule that recognizes the MHC-target peptide complex when a) the percentile rank of a mutated peptide with a mutation associated with the respective position is less than 1.0 if the percentile rank of the target peptide is less than or equal to 0.5, b) the percentile rank of a mutated peptide with a mutation associated with the respective position is less than 0 if the percentile rank of the target peptide is between 0.5 and 0, or c) the percentile rank of a mutated peptide with a mutation associated with the respective position is less than 4.0 if the percentile rank of the target peptide is greater than or equal to 0, as determined by the percentile rank value. Implementations of the described techniques may include hardware, a method or process, or computer software on a computer-accessible medium.

One general aspect includes non-transitory computer-readable medium configured to communicate with one or more processor(s) of a computational device. The non-transitory computer-readable medium includes instructions to perform the following steps, which can be performed in various orders and with interleaving steps: a) receive, as an input, a computational representation of a target peptide presented in a MHC-target peptide complex; b) predict all amino acid positions within the target peptide that may be involved in interacting with an antigen-recognition molecule that recognizes the MHC-target peptide complex; d) generate a working list of peptides, within a total pool of predicted or detected peptides of suitable length, such that peptides listed in the working list each include at least two amino acids that (i) are located at positions corresponding to positions within the target peptide that are involved in interacting with the antigen-recognition molecule and (ii) are identical to the corresponding amino acids of the target peptide; e) determine binding affinity, of each of the peptides listed in the working list, to the MHC molecule of the MHC-target peptide complex; f) filter the working list to include only peptide(s) having a calculated binding affinity to the MHC molecule greater than a first threshold value, thereby generating a working list of off-target peptide(s); and g) provide, as an output, the working list of off-target peptide(s) and/or the number of the off-target peptide(s) in the working list. Other embodiments of this aspect include corresponding computer systems, apparatus, and computer programs recorded on one or more computer storage devices, each configured to perform the actions of the methods.

Implementations may include one or more of the following features. The non-transitory computer-readable medium may include instructions that, when executed by the processor(s), cause the computational device to: estimate the number of peptide(s) in the working list of off-target peptides which are expressed in essential, normal tissues; and provide, as an output, the number of off-target peptide(s) which are expressed in essential, normal tissues. The instructions, when executed by the processor(s), cause the computational device to: determine, for each peptide in the working list of peptides, whether such peptide is expressed in essential, normal tissues; and filter the working list to include only peptide(s) which are expressed in essential, normal tissues. The instructions, when executed by the processor(s), cause the computational device to: generate a list of potential secondary target peptides comprising peptides having a calculated binding affinity to the MHC molecule greater than the first threshold value and having low expression in essential, normal tissues. The instructions, when executed by the processor(s), cause the computational device to: calculate a degree of similarity (DOS) score for the peptide(s) in the working list of peptides, the DoS score being based at least in part on a number of amino acids identical to amino acids at corresponding positions of the target peptide, which amino acids of the target peptide are involved in interacting with the antigen-recognition molecule; and filter the working list to include only peptide(s) having a DoS score greater than a second threshold value. Only positions of the target peptide identified as being unbound to the MHC molecule are considered in calculating the DoS score. The instructions, when executed by the processor(s), cause the computational device to: provide, as an input, a computational representation of the antigen-recognition molecule, the antigen-recognition molecule being capable of binding to the MHC-target peptide complex; determine binding affinity of the antigen-recognition molecule to a plurality of MHC-peptide complexes each including a respective likely off-target peptide from the working list and the MHC molecule; and filter the working list to include only likely off-target peptides which may include a binding motif for the antigen-recognition molecule. The instructions, when executed by the processor(s), cause the computational device to: provide, as an input, off-target peptide expression in essential, normal tissues of a specific patient; and provide, as an output, an indication of the off-target effects for said patient. Implementations of the described techniques may include hardware, a method or process, or computer software on a computer-accessible medium.

One general aspect includes non-transitory computer-readable medium configured to communicate with one or more processor(s) of a computational device. The non-transitory computer-readable medium includes instructions to perform the following steps, which can be performed in various orders and with interleaving steps: a) receive, as an input, a computational representation of a target peptide presented in a complex with a major histocompatibility complex (MHC) molecule (MHC-target peptide complex); b) identify, within a total pool of predicted or detected peptides of suitable length, similar peptides that include at least two amino acids that (i) are located at positions corresponding to positions within the target peptide that are involved in interacting with an antigen-recognition molecule and (ii) are identical to the corresponding amino acids of the target peptide; c) determine binding affinity of each of the identified similar peptide(s) to the MHC molecule; d) identify off-target peptide(s) based at least in part on identifying similar peptide(s) having a calculated binding affinity to the MHC molecule stronger than a first threshold value; and e) provide, as an output, the off-target peptide(s).

One general aspect includes non-transitory computer-readable medium configured to communicate with one or more processor(s) of a computational device. The non-transitory computer-readable medium includes instructions to perform the following steps, which can be performed in various orders and with interleaving steps: a) select two or more potential target peptides, among disease-associated peptides, that are predicted to bind to a MHC molecule; b) estimate a number of off-target peptide(s) associated with each of the potential target peptides, and c) rank the potential target peptides based at least in part on the number of off-target peptide(s) associated with each of the potential target peptides. Other embodiments of this aspect include corresponding computer systems, apparatus, and computer programs recorded on one or more computer storage devices, each configured to perform the actions of the methods.

Implementations may include one or more of the following features. The non-transitory computer-readable medium may include instructions that, when executed by the processor(s), cause the computational device to: calculate a DoS score for each of the off-target peptides such that DoS score represents similarities between a respective off-target peptide and the target peptide; and rank the potential target peptides based at least in part on the DoS score(s) of off-target peptide(s) associated with each of the potential target peptides. The instructions, when executed by the processor(s), cause the computational device to: calculate the DoS score based at least in part on a number of amino acids of the off-target peptide identical to amino acids at corresponding positions of the target peptide, which amino acids of the target peptide are involved in interacting with an antigen-recognition molecule. Only positions of the target peptide identified as not involved in interacting with the MHC molecule are considered in calculating the DoS score. The instructions, when executed by the processor(s), cause the computational device to: calculate a probability of in vivo toxicity of each potential target peptide based at least in part on the DoS scores of the off-target peptide(s). The probability of in vivo toxicity of each potential target peptide is based at least in part on a number of high-toxicity off-target peptide(s) that have a DoS score above a predetermined threshold value. The disease-associated peptides in step (a) are identified based at least in part on comparison of the level of expression of the corresponding mRNA or protein in disease-affected tissue(s) and essential, normal tissue(s). Implementations of the described techniques may include hardware, a method or process, or computer software on a computer-accessible medium.

One general aspect includes non-transitory computer-readable medium including a database thereon, the database including a plurality of disease-associated peptide sequences each associated with off-target peptides and ranked according to probability of in vivo toxicity related to the respective off-target peptides, the off-target peptides each having an approximately equal binding affinity to a MHC molecule as a peptide identified by the respective disease-associated peptide sequence. Other embodiments of this aspect include corresponding computer systems, apparatus, and computer programs recorded on one or more computer storage devices, each configured to perform the actions of the methods.

One general aspect includes non-transitory computer-readable medium including a database thereon, the database including a plurality of disease-associated peptide sequences; and a plurality of off-target peptide sequences each associated with a respective disease-associated peptide sequence, where each disease-associated peptide identified by the disease-associated peptide sequences has a binding affinity to a MHC molecule that is approximately equal to binding affinity of off-target peptides identified by the respective off-target peptide sequences to the MHC molecule. Other embodiments of this aspect include corresponding computer systems, apparatus, and computer programs recorded on one or more computer storage devices, each configured to perform the actions of the methods.

One general aspect relates to a method for predicting amino acid position(s) within a target peptide presented in a MHC-target peptide complex, the amino acid position(s) being involved in interacting with an antigen-recognition molecule that recognizes said MHC-target peptide complex, the method comprising: a) determining binding affinity of the target peptide to the MHC molecule; b) generating a plurality of mutated peptides each associated with a mutation at a respective amino acid position of the target peptide; c) determining binding affinity of each mutated peptide of the plurality of mutated peptides to the MHC molecule; and d) predicting the amino acid position(s) involved in interacting with the antigen-recognition molecule based at least in part on a comparison of the binding affinity for each mutated peptide to the binding affinity of the target peptide.

In certain embodiments, the binding affinity of the target peptide to the MHC molecule is determined using a computer-based model.

In certain embodiments, the binding affinity of the target peptide to the MHC molecule is determined through an experimental measurement.

In certain embodiments, the binding affinity of the target peptide to the MHC molecule is determined using the half maximal inhibitory concentration (IC₅₀) value or a percentile rank value.

In certain embodiments, generating a plurality of mutated peptides involves mutating the respective amino acid to a glycine amino acid.

In certain embodiments, generating a plurality of mutated peptides involves mutating the respective amino acid to an alanine amino acid.

In certain embodiments, the method further includes predicting that a respective position of the plurality of positions is involved in interacting with an antigen-recognition molecule that recognizes the MHC-target peptide complex when the binding affinity of a mutated peptide with a mutation associated with the respective position is approximately equal to the binding affinity of the target peptide.

In certain embodiments, the method further includes predicting that a respective position of the plurality of positions is involved in interacting with an antigen-recognition molecule that recognizes the MHC-target peptide complex when a) the percentile rank of a mutated peptide with a mutation associated with the respective position is less than 1.0 if the percentile rank of the target peptide is less than or equal to 0.5, b) the percentile rank of a mutated peptide with a mutation associated with the respective position is less than 2.0 if the percentile rank of the target peptide is between 0.5 and 2.0, or c) the percentile rank of a mutated peptide with a mutation associated with the respective position is less than 4.0 if the percentile rank of the target peptide is greater than or equal to 2.0, as determined by the percentile rank value.

In certain embodiments, the method further includes predicting that a respective position of the plurality of positions is involved in interacting with an antigen-recognition molecule that recognizes the MHC-target peptide complex when both of the following conditions are met: (i) the binding affinity of a mutated peptide with a mutation associated with the respective position is approximately equal to the binding affinity of the target peptide, and (ii) the amino acid of the target peptide at the respective position is a non-glycine residue.

In certain embodiments, the method further includes verifying that that the respective position of the plurality of positions within the target peptide is not involved in interacting with the MHC molecule based on a known structure of the MHC-target peptide complex.

One general aspect relates to a method of identifying off-target peptide(s) for an antigen-recognition molecule that recognizes a target peptide presented in a complex with a major histocompatibility complex (MHC) molecule (MHC-target peptide complex), the method comprising: a) predicting all amino acid positions within the target peptide that may be involved in interacting with the antigen-recognition molecule; b) identifying within a total pool of predicted or detected peptides of suitable length similar peptides that include at least two amino acids that (i) are located at positions corresponding to positions within the target peptide that are involved in interacting with the antigen-recognition molecule and (ii) are identical to the corresponding amino acids of the target peptide; c) determining binding affinity of each of the identified similar peptide(s) to the MHC molecule; and d) identifying off-target peptide(s) based at least in part on identifying similar peptide(s) having a calculated binding affinity to the MHC molecule stronger than a first threshold value.

One general aspect relates to a method for estimating a number of off-target peptide(s) for an antigen-recognition molecule that recognizes a target peptide presented in a MHC-target peptide complex, the method comprising: a) predicting all amino acid positions within the target peptide that may be involved in interacting with the antigen-recognition molecule; b) identifying within a total pool of predicted or detected peptides of suitable length similar peptides that include at least two amino acids that (i) are located at positions corresponding to positions within the target peptide that are involved in interacting with the antigen-recognition molecule and (ii) are identical to the corresponding amino acids of the target peptide; c) determining binding affinity of each of the identified similar peptide(s) to the MHC molecule; and d) estimating the number of off-target peptide(s) based at least in part on counting similar peptide(s) having a calculated binding affinity to the MHC molecule stronger than a first threshold value.

In certain embodiments, step (b) comprises identifying within a total pool of predicted or detected peptides of suitable length similar peptides that include at least three amino acids that (i) are located at positions corresponding to positions within the target peptide that are involved in interacting with the antigen-recognition molecule and (ii) are identical to the corresponding amino acids of the target peptide.

In certain embodiments, the step (b) comprises identifying within a total pool of predicted or detected peptides of suitable length similar peptides that include at least four amino acids that (i) are located at positions corresponding to positions within the target peptide that are involved in interacting with the antigen-recognition molecule and (ii) are identical to the corresponding amino acids of the target peptide.

In certain embodiments, the step (b) comprises identifying within a total pool of predicted or detected peptides of suitable length similar peptides that include at least five amino acids that (i) are located at positions corresponding to positions within the target peptide that are involved in interacting with the antigen-recognition molecule and (ii) are identical to the corresponding amino acids of the target peptide.

In certain embodiments, the binding affinity in step (c) is determined by determining the percentile rank value, and wherein the number of off-target peptide(s) is estimated in step (d) based at least in part on counting similar peptide(s) having percentile rank value lower than 2.0.

In certain embodiments, the method further includes including in the off-target peptide(s) only peptide(s) which are expressed in essential, normal tissues.

In certain embodiments, the method further includes determining, for each of the identified off-target peptide(s), whether such peptide is expressed in essential, normal tissues.

In certain embodiments, the peptide expression is determined based on the level of the corresponding mRNA or protein expression.

In certain embodiments, the peptide expression is determined based on mass-spectrometry data.

In certain embodiments, the method further includes including in the off-target peptide(s) only similar peptide(s) having the calculated binding affinity to the MHC molecule stronger than the first threshold value and detectable expression in essential, normal tissues.

In certain embodiments, the method further includes calculating a Degree of Similarity (DoS) score for the similar peptide(s), the DoS score being based at least in part on a number of amino acids of a respective similar peptide identical to amino acids at corresponding positions of the target peptide, which amino acids of the target peptide are involved in interacting with the antigen-recognition molecule.

In certain embodiments, only positions of the target peptide identified as being unbound to the MHC molecule are considered in calculating the DoS score.

In certain embodiments, the method further includes including in the off-target peptide(s) only similar peptide(s) having a DoS score greater than a second threshold value.

In certain embodiments, predicting amino acid positions within the target peptide that are involved in interacting with the antigen-recognition molecule in step (a) is conducted using the method for estimating a number of off-target peptide(s) for an antigen-recognition molecule that recognizes a target peptide presented in a MHC-target peptide complex disclosed supra.

In certain embodiments, the total pool of detected peptides in step (b) is based on mass-spectrometry data.

In certain embodiments, the MHC molecule is a class I MHC molecule, and the predicted or detected peptides in step (b) are 8-12 amino acids in length.

In certain embodiments, the MHC molecule is a class I MHC molecule, and the target peptide is 8-12 amino acids in length.

In certain embodiments, the antigen-recognition molecule is a T cell Receptor (TCR), a chimeric antigen receptor (CAR), an antibody, or an antigen-binding fragment thereof.

One general aspect relates to a method for ranking potential target peptides to mitigate off-target toxicity, the method comprising: a) selecting two or more potential target peptides among disease-associated peptides that are predicted to bind to an MHC molecule; b) estimating a number of off-target peptide(s) associated with each of the potential target peptides; and c) ranking the potential target peptides based at least in part on the number of off-target peptide(s) associated with each of the potential target peptides.

In certain embodiments, the number of off-target peptide(s) in step (b) is estimated using the method described herein.

In certain embodiments, the method further includes ranking the potential target peptides such that potential target peptides having fewer associated off-target peptide(s) are selected for further analysis and/or are used for generation and/or testing of an antigen-recognition molecule(s).

In certain embodiments, the method further includes calculating a Degree of Similarity (DoS) score for each of the off-target peptides such that DoS score represents similarities between a respective off-target peptide and the target peptide; and ranking the potential target peptides based at least in part on the DoS score(s) of off-target peptide(s) associated with each of the potential target peptides.

In certain embodiments, calculating the DoS score is based at least in part on a number of amino acids of the off-target peptide identical to amino acids at corresponding positions of the target peptide, which amino acids of the target peptide are involved in interacting with an antigen-recognition molecule.

In certain embodiments, only positions of the target peptide identified as not involved in interacting with the MHC molecule are considered in calculating the DoS score.

In certain embodiments, the method further includes calculating a probability of in vivo toxicity of each potential target peptide based at least in part on the DoS scores of the off-target peptide(s).

In certain embodiments, the probability of in vivo toxicity of each potential target peptide is based at least in part on a number of high-toxicity off-target peptide(s) that have a DoS score above a predetermined threshold value.

In certain embodiments, the method further includes ranking the target peptides such that potential target peptides having lower toxicity are prioritized.

In certain embodiments, the disease-associated peptides in step (a) are identified based at least in part on comparison of the level of expression of the corresponding mRNA or protein in disease-affected tissue(s) and essential, normal tissue(s).

In certain embodiments, the method further includes providing the ranking of the potential target peptides in a database.

In certain embodiments, the method further includes providing a list of the off-target peptide(s) associated with each of the potential target peptides in a database.

In another aspect, provided herein is an in vitro method of assessing off-target effects of an antigen-recognition molecule, comprising a) contacting the antigen-recognition molecule with a target peptide presented in a complex with a major histocompatibility complex (MHC) molecule (MHC-target peptide complex); b) contacting the antigen-recognition molecule with one or more off-target peptides associated with said target peptide, wherein each of said off-target peptides is presented in a complex with the same MHC molecule as in (a) (MHC-off-target peptide complex); and c) determining and comparing the level of binding of the antigen-recognition molecule to MHC-target peptide complex and each of the MHC-off-target peptide complexes.

In another aspect, provided herein is an in vitro method of assessing off-target effects of an antigen-recognition molecule, comprising a) contacting the antigen-recognition molecule with one or more off-target peptides associated with a target peptide that is recognized by the antigen-recognition molecule, wherein each of said off-target peptides is presented in a complex with a major histocompatibility complex (MHC) molecule (MHC-off-target peptide complex); and b) determining the level of binding of the antigen-recognition molecule to each of the MHC-off-target peptide complexes.

In some embodiments of the in vitro methods described above, the method may further comprise determining that the antigen-recognition molecule is likely to have off-target effects if it detectably binds to at least one MHC-off-target peptide complex, wherein the off-target peptide is expressed in essential, normal tissues.

In another aspect, provided herein is a method for selecting an antigen-recognition molecule, comprising: a) contacting a plurality of antigen-recognition molecules with a target peptide presented in a complex with a major histocompatibility complex (MHC) molecule (MHC-target peptide complex); b) contacting the same plurality of antigen-recognition molecules with one or more off-target peptides associated with said target peptide, wherein each of said off-target peptides is presented in a complex with the same MHC molecule as in (a) (MHC-off-target peptide complex); c) selecting one or more antigen-recognition molecules based at least in part on the number of MHC-off-target peptide complexes detectably bound by each of the antigen-recognition molecules; and d) optionally, repeating steps (a)-(c) using the one or more selected antigen-recognition molecules.

In some embodiments, the one or more selected antigen-recognition molecules detectably bind no more than five (e.g., no more than four, no more than three, no more than two, or no more than one) MHC-off-target peptide complexes, wherein the off-target peptides are expressed in essential, normal tissues.

In some embodiments, the one or more selected antigen-recognition molecules do not detectably bind to any of the MHC-off-target peptide complexes, wherein the off-target peptides are expressed in essential, normal tissues.

In some embodiments, the plurality of antigen-recognition molecules is in a library. In some embodiments, the library is a phage display library or a yeast library.

In various embodiments, the MHC-peptide complexes are immobilized on a solid support. In various embodiments, the MHC-peptide complexes are present on antigen-presenting cells.

In various embodiments, the level of binding is determined by detecting the amount of antigen-recognition molecules bound to the MHC-peptide complexes.

In various embodiments, the method is performed in a high-throughput format (e.g., a 96-well plate).

In another aspect, provided herein is a method of enriching a sample for antigen-recognition molecules that specifically bind a target peptide, comprising a) contacting a sample comprising a plurality of antigen-recognition molecules with the target peptide in the presence of one or more off-target peptides associated with said target peptide, wherein each of said target peptide and said one or more off-target peptides is presented in a complex with a major histocompatibility complex (MHC) molecule (MHC-target peptide complex or MHC-off-target peptide complex); b) enriching the sample by isolating the antigen-recognition molecules that are bound to the MHC-target peptide complex; and c) optionally, repeating steps (a)-(b) using the enriched sample.

In some embodiments of the method described above, the MHC-target peptide complex is present on antigen-presenting cells and the MHC-off-target peptide complexes are not present on antigen-presenting cells. In some embodiments, the MHC-target peptide complex is immobilized on a solid support and the MHC-off-target peptide complexes are not immobilized on the solid support. In some embodiments, the MHC-target peptide complex is labeled, and the MHC-off-target peptide complexes are not labeled or differentially labeled as said MHC-target peptide complex.

In various embodiments of the methods described above, the one or more off-target peptides are identified using the method described herein.

In various embodiments of the methods described above, the antigen-recognition molecule is a T cell Receptor (TCR), a chimeric antigen receptor (CAR), an antibody, or an antigen-binding fragment thereof. In some embodiments, the antigen-recognition molecule is present in a solution. In some embodiments, the antigen-recognition molecule is present on a cell.

In a yet further aspect, provided herein is a library comprising at least two off-target peptides identified using the method described herein. In some embodiments, a library of the present disclosure comprises one or more peptides each selected from the amino acid sequences of SEQ ID NOs: 1-7, 9, and 11-74, or any combination thereof. In some embodiments, the at least two off-target peptides are each present in a complex with a major histocompatibility complex (MHC) molecule.

### BRIEF DESCRIPTION OF THE DRAWINGS

The above and further aspects of this invention should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

FIG. 1A illustrates a flow diagram of an exemplary embodiment of a Peptide in Groove Similarity Prediction (PIGSPRED) method.

FIG. 1B illustrates a flow diagram of an exemplary embodiment of a method for predicting amino acid position(s) within a target peptide presented in a complex with a major histocompatibility complex (MHC) molecule (MHC-target peptide complex) that can be performed as part of the PIGSPRED method.

FIG. 2 illustrates a flow diagram of an embodiment of a method to compute expected off-target toxicity associated with an MHC-target peptide complex.

FIG. 3 illustrates a flow diagram of an embodiment of a method for ranking potential target peptides to mitigate off-target toxicity.

FIG. 4 illustrates a block diagram of an exemplary embodiment of a computational system configured to output an off-target peptide list and/or an off-target toxicity metric, the system including an exemplary embodiment of a PIGSPRED engine.

FIG. 5 illustrates a block diagram of an exemplary embodiment of a computations system configured to output a list of low risk peptide targets, off-target peptide list(s) for potential target(s), and/or off-target toxicity metric(s) for potential target(s), the system including an exemplary embodiment of a target ranking engine.

FIG. 6 illustrates a block diagram of an exemplary embodiment of a target toxicity database.

FIG. 7 illustrates a block diagram of an embodiment of a computing device.

FIG. 8 illustrates a block diagram of an embodiment of a computing network.

FIG. 9 illustrates cellular functions related to the example embodiments presented herein.

### DEFINITIONS

Unless defined otherwise, all technical and scientific terms used herein have the same meaning as commonly understood by one skilled in the pertinent art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a meaningful range of a value. The allowable variation encompassed by the term “about” or “approximately” depends on the particular system under study, and can be readily appreciated by one skilled in the pertinent art.

The terms “major histocompatibility complex,” and “MHC” encompass the terms “human leukocyte antigen” or “HLA” (the latter two of which are generally reserved for human MHC molecules), naturally occurring MHC molecules (e.g., MHC class I molecule comprising MHC class I α (heavy) chain and β2 microglobulin; MHC class II molecule comprising MHC class II α chain and MHC class II β chain), individual chains of MHC molecules (e.g., MHC class I α (heavy) chain, MHC class II α chain, and MHC class II β chain), individual subunits of such chains of MHC molecules (e.g., α1, α2, and/or α3 subunits of MHC class I α chain, α1-α2 subunits of MHC class II α chain, β1-β2 subunits of MHC class II B chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a T-cell receptor (TCR), e.g., an antigen-specific TCR. An MHC class I molecule comprises a peptide binding groove formed by the α1 and α2 domains of the heavy a chain that can stow a peptide of around 8-10 amino acids. Despite the fact that both classes of MHC bind a core of about 9 amino acids (e.g., 5 to 17 amino acids) within peptides, the open-ended nature of MHC class II peptide binding groove (the α1 domain of a class II MHC a polypeptide in association with the β1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time. In some embodiments, the MHC-peptide complex described herein may be an MHC-peptide complex from a non-human animal. In other embodiments, the MHC-peptide complex described herein may include an HLA-peptide complex, i.e., an MHC-peptide complex from a human.

The term “non-human animal” and the like refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human animal is a mammal. In some embodiments, a non-human mammal is a primate, a goat, a sheep, a pig, a dog, a cow, or a rodent. In some embodiments, a non-human animal is a rodent such as a rat or a mouse.

The term “antigen” refers to any agent (e.g., protein, peptide, polysaccharide, glycoprotein, glycolipid, nucleotide, portions thereof, or combinations thereof) that, when introduced into an immunocompetent host is recognized by the immune system of the host and elicits an immune response by the host. The T-cell receptor recognizes a peptide presented in the context of a major histocompatibility complex (MHC) as part of an immunological synapse. The peptide-MHC (pMHC) complex is recognized by TCR, with the peptide (antigenic determinant) and the TCR idiotype providing the specificity of the interaction. Accordingly, the term “antigen” encompasses peptides presented in the context of MHCs, e.g., peptide-MHC complexes. The peptide displayed on MHC may also be referred to as an “epitope” or an “antigenic determinant”. The terms “peptide,” “antigenic determinant,” “epitopes,” etc., encompass not only those presented naturally by antigen-presenting cells (APCs), but may be any desired peptide so long as it is recognized by an immune cell of an animal, e.g., when presented appropriately to the cells of an immune system. For example, a peptide having an artificially prepared amino acid sequence may also be used as the epitope.

The term “antigen-recognition molecule” refers to any molecule that is capable of recognizing an antigen as defined above. Antigen-recognition molecules can include, but are not limited to, T cell receptors (TCR), antibodies, antibody fragments, or chimeric antigen receptors (CARs).

“MHC-peptide complex,” “peptide-MHC complex,” “pMHC complex,” “peptide-in-groove,” and the like includes
(i) an MHC molecule, e.g., a human and/or non-human animal MHC molecule, or portion thereof (e.g., the peptide-binding groove thereof, and e.g., the extracellular portion thereof), and
(ii) an antigenic peptide, where the MHC molecule and the antigenic peptide are complexed in such a manner that the pMHC complex can specifically bind a T-cell receptor. A pMHC complex encompasses cell surface expressed pMHC complexes and soluble pMHC complexes.

“HLA-peptide complex,” “peptide-HLA complex,” “pHLA complex,” and the like refers to an MHC-peptide complex wherein the MHC molecule is a Human Leukocyte Antigen (HLA) molecule.

The terms “antibody,” “antibodies,” “immunoglobulin, “binding protein” and the like refer to monoclonal antibodies, multispecific antibodies, human antibodies, humanized antibodies, chimeric antibodies, single-chain Fvs (scFv), single chain antibodies, Fab fragments, F (ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 20070004909, incorporated herein by reference in its entirety, and Ig-DARTS such as those disclosed in U.S. Pat. Appl. Pub. 20090060910, incorporated herein by reference in its entirety. A pMHC-binding protein refers to an antigen-binding protein, immunoglobulin, antibody, or the like that specifically binds a pMHC complex.

An “individual” or “subject” or “animal” refers to humans, veterinary animals (e.g., cats, dogs, cows, horses, sheep, pigs, etc.) and experimental animal models of diseases (e.g., mice, rats). In one embodiment, the subject is a human.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid” and “nucleotide” encompass both DNA and RNA unless specified otherwise.

The term “library” refers to an isolated collection of at least two elements that differ from one another in at least one aspect. For example, a “peptide library” is a collection of at least two peptides that may differ from one another by at least one amino acid. As another example, a “pMHC complex library” is a collection of pMHC complexes that may differ from one another by at least one amino acid in the peptide or at least one MHC polypeptide. The elements of the library are isolated from like type of elements that are not part of the library (e.g., peptides of a peptide library are isolated from peptides that are not part of the library). The library may exist in vitro or ex vivo.

The term “administration” and the like refers to and includes the administration of a composition (e.g., antigen-recognition molecule) to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

The term “essential, normal tissues” refers to tissues of a patient where an activity of a given antigen-recognition molecule administered for treating a disease may create unacceptable side-effects. The list of tissues considered essential, normal would vary depending on the disease being treated and on the risks associated with the disease itself (e.g., the list would be smaller for life-threatening diseases than for non-life-threatening diseases). For example but not by way of limitation, when treating life-threatening cancers, tissue types that may be considered non-essential may include breast, ovary and testes. The list of tissues considered essential, normal would also vary depending on the likelihood for a given antigen-recognition molecule to reach such tissues. For example, brain may not be included in the list of essential, normal tissues in cases of antigen-recognition molecules which do not permeate blood-brain-barrier of patients with the disease being treated.

The terms “component,” “engine,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

The term “connected” means that one function, feature, structure, or characteristic is directly joined to or in communication with another function, feature, structure, or characteristic.

The term “coupled” means that one function, feature, structure, or characteristic is directly or indirectly joined to or in communication with another function, feature, structure, or characteristic.

The terms “comprising” or “containing” or “including” are meant that at least the named element, or method step is present in article or method, but does not exclude the presence of other elements or method steps, even if the other such elements or method steps have the same function as what is named.

As used herein, unless otherwise specified, the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

In this description, numerous specific details are set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

### DETAILED DESCRIPTION

Some embodiments presented herein relate to identification of off-target peptide(s) that are similar to an intended target peptide of an MHC-target peptide complex, such that an antigen-recognition molecule that is engineered for the intended target MHC-target peptide complex is likely to also target the off-target peptide(s). In some examples, identification of the off-target peptide(s) can include predicting amino acid position(s) within the target peptide presented in the MHC-target peptide complex that are available to be involved in interacting with an antigen-recognition molecule. In some embodiments, the identified off-target peptide(s) can be used to predict probability of in vivo toxicity of a treatment targeting the target peptide. In some embodiments, off-target peptides can be identified for several potential target peptides, and the target peptides can be ranked based, at least in part, on their associated off-target peptides. In some embodiments, the identification of the off-target peptide(s) can be agnostic of the antigen-recognition molecule so that the identified off-target peptides, predicted in vivo toxicity, and/or ranking of potential target peptides can be used to guide development of an antigen-recognition molecule engineered to target a target peptide having a low number of identified off-target peptides, a low predicted probability of in vivo toxicity, and/or a preferred ranking. Some embodiments disclosed herein include computational systems, engines, modules, devices, and/or networks configured to carry out a majority of steps associated with the above embodiments. The output of such computational systems, etc. can be used to inform antigen-recognition molecule development, screening of patients for clinical trials, individual patient treatment, and other such applications as understood by a person skilled in the pertinent art according to the teachings herein. One aim of some embodiments presented herein is to avoid side effects that would otherwise be identified during clinical trials, thereby reducing patient death, reducing other adverse effects on patients, and reducing expenditure of time and resources in research and development.

MHC molecules are generally classified into two categories: class I and class II MHC molecules. An MHC class I molecule is an integral membrane protein comprising a glycoprotein heavy chain, also referred to herein as the a chain, which has three extracellular domains (i.e., α1, α2 and α3) and two intracellular domains (i.e., a transmembrane domain (TM) and a cytoplasmic domain (CYT)). The heavy chain is noncovalently associated with a soluble subunit called β2 microglobulin (β2m or β2M). An MHC class II molecule or MHC class II protein is a heterodimeric integral membrane protein comprising one a chain and one β chain in noncovalent association. The a chain has two extracellular domains (α1 and α2), and two intracellular domains (a TM domain and a CYT domain). The β chain contains two extracellular domains (β1 and β2), and two intracellular domains (a TM domain and CYT domain).

The domain organization of class I and class II MHC molecules forms the antigenic determinant binding site, e.g., the peptide-binding portion or peptide binding groove, of the MHC molecule. A peptide binding groove refers to a portion of an MHC protein that forms a cavity in which a peptide, e.g., antigenic determinant, can bind. The conformation of a peptide binding groove is capable of being altered upon binding of a peptide to enable proper alignment of amino acid residues important for TCR binding to the peptide-MHC (pMHC) complex.

In some embodiments, MHC molecules include fragments of MHC chains that are sufficient to form a peptide binding groove. For example, a peptide binding groove of a class I protein can comprise portions of the α1 and α2 domains of the heavy chain capable of forming two B-pleated sheets and two a helices. Inclusion of a portion of the β2 microglobulin chain stabilizes the MHC class I molecule. While for most versions of MHC Class II molecules, interaction of the a and B chains can occur in the absence of a peptide, the two-chain molecule of MHC Class II is unstable until the binding groove is filled with a peptide. A peptide binding groove of a class II protein can comprise portions of the α1 and β1 domains capable of forming two β-pleated sheets and two a helices. A first portion of the α1 domain forms a first β-pleated sheet and a second portion of the α1 domain forms a first a helix. A first portion of the β1 domain forms a second β-pleated sheet and a second portion of the β1 domain forms a second a helix. The X-ray crystallographic structure of class II protein with a peptide engaged in the binding groove of the protein shows that one or both ends of the engaged peptide can project beyond the MHC protein (Brown et al., pp. 33-39, 1993, Nature, Vol. 364; incorporated herein in its entirety by reference). Thus, the ends of the α1 and β1 α helices of class II form an open cavity such that the ends of the peptide bound to the binding groove are not buried in the cavity. Moreover, the X-ray crystallographic structure of class II proteins shows that the N-terminal end of the MHC β chain apparently projects from the side of the MHC protein in an unstructured manner since the first 4 amino acid residues of the β chain could not be assigned by X-ray crystallography. Many human and other mammalian MHCs are well known in the art.

In some embodiments, the MHC molecule may be a human HLA molecule selected from the group consisting of HLA-A, HLA-B, HLA-C, HLA-E, HLA-F, and HLA-G. A list of commonly used HLA alleles is described in Shankarkumar et al. ((2004) The Human Leukocyte Antigen (HLA) System, Int. J. Hum. Genet. 4 (2): 91-103), incorporated herein in its entirety by reference. Shankarkumar et al. also present a brief explanation of HLA nomenclature used in the art. Additional information regarding HLA nomenclature and various HLA alleles can be found in Holdsworth et al. (2009) The HLA dictionary 2008: a summary of HLA-A, -B, -C, -DRB1/3/4/5, and DQB1 alleles and their association with serologically defined HLA-A, -B, -C, -DR, and -DQ antigens, Tissue Antigens 73:95-170, and a recent update by Marsh et al. (2010) Nomenclature for factors of the HLA system, 2010, Tissue Antigens 75:291-455, each of which publications is incorporated herein in its entirety by reference. In some embodiments, the MHC I or MHC II polypeptides may be derived from any functional human HLA-A, B, C, DR, or DQ molecules. In one embodiment, the HLA molecule is encoded by HLA-A2, such as an HLA-A*02:01 allele. In another embodiment, the HLA molecule is encoded by HLA-A1, such as an HLA-A*01:01 allele.

Targeting peptide-MHC (pMHC) complexes specifically expressed on cells such as cancer cells via, e.g., antibody-based or cell-based therapeutics approaches, can be an effective way of destroying such cells. However, the potential off-targets associated with these pMHC complexes can often lead to off-target toxicity. The present disclosure provides, among other things, a method named PIGSPRED (Peptide in Groove Similarity Prediction) useful in the prediction of such off-targets.

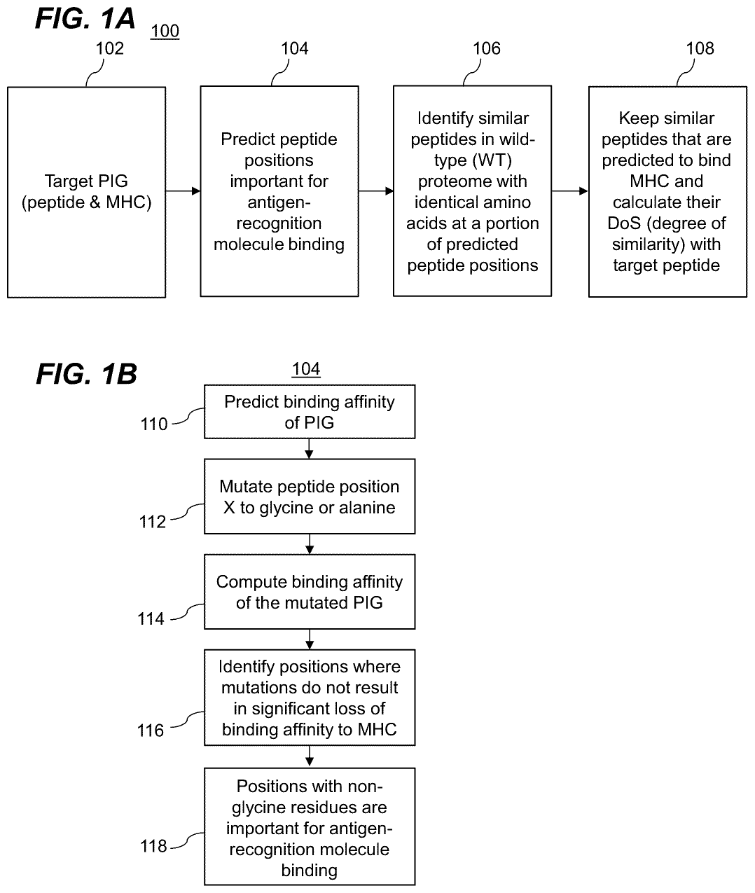

FIG. 1A illustrates a flow diagram of an exemplary embodiment of a Peptide in Groove Similarity Prediction (PIGSPRED) method 100.

At step 102, an MHC-target peptide complex is given as an input to the PIGSPRED method. The MHC-target peptide complex includes a target peptide and an MHC molecule.

At step 104, peptide positions (amino acids) important for antigen-recognition molecule binding are identified. The target peptide includes some amino acids which are bound to the MHC molecule and some amino acids which are available for binding to an antigen-recognition molecule. When evaluating similarity/homology of a peptide to the target peptide, the similarity can be evaluated only at peptide positions (i.e., positions of amino acids within the peptide) that are available to bind with an antigen-recognition molecule. These available positions can be ascertained by analyzing the experimental structures of the MHC-target peptide complex with a specific antigen-recognition molecule, wherein such experimental structures are typically derived using crystallography or cryoEM techniques. Therefore, peptide positions important for antigen-recognition molecule binding can be identified through analysis of experimental structures; however, the specific antigen-recognition molecule must be known and such experimental structures can be difficult to obtain. When developing a targeted therapy, at the initial target selection stage, in which the potential risks associated with the target are evaluated, a specific antigen-recognition molecule against the target is unavailable. The Immune Epitope Database (IEDB) is a freely available resource that catalogs experimental data on antibody and T cell epitopes studied in humans, non-human primates, and other animal species in the context of infectious disease, allergy, autoimmunity and transplantation. The IEDB also hosts tools to assist in the prediction and analysis of epitopes. Similar databases and methodologies to characterize epitopes in such databases can be used to obtain experimental structures of MHC peptide complexes.

FIG. 1B illustrates steps of the PIGSPRED method 100 that can be performed at step 104. The steps illustrated in FIG. 1B illustrate an exemplary embodiment of a method 104 for predicting amino acid position(s) within the target peptide presented in the MHC-target peptide complex. The method 104 can be performed agnostic to structure of an antigen-recognition molecule, and therefore presents an alternative to analysis of the experimental structures of the MHC-target peptide complex with a specific antigen-recognition molecule when the specific antigen-recognition molecule is unknown. Positions identified computationally at step 104 can be verified by experimental data such as mass-spectroscopy data or experimental data otherwise available in a database similar to the IEDB.

At step 110, binding affinity of the MHC molecule to the target peptide is predicted. In some embodiments, the binding affinity can be calculated using a computer-based model. As a non-limiting example, a commercial tool called NetMHCpan, which uses a machine learning model, utilizes a computer-based binding affinity model that may be suitable for computationally calculating the binding affinity. Any version of NetMHCpan may be used, for example, NetMHCpan4.0 or NetMHCpan4.1. Other non-limiting examples include MHCflurry (see, e.g., O'Donnell et al., Cell Syst. 2018 Jul. 25; 7(1): 129-132.e4, which is incorporated herein by reference in its entirety) and MixMHCPred (see, e.g., Boehm et al., BMC Bioinformatics volume 20, Article number: 7 (2019), which is incorporated herein by reference in its entirety).

Predicting binding affinity estimates how tightly a peptide will bind to a specific MHC molecule. It is measured as either a predicted IC₅₀ value in nanomolar (nM) range or a predicted percentile rank value. The predicted IC₅₀ value attempts to approximate experimental IC₅₀ value which measures the concentration of a competing peptide required to displace 50% of the binding of the peptide to the MHC molecule. In some embodiments, if the predicted binding affinity≤500 nM, a peptide is considered a binder. If it is ≤50 nM, it is considered a strong binder. The percentile rank value (e.g., % Rank_BA in NetMHCpan) normalizes the IC₅₀ values across different MHC molecules. The rank is computed by comparing the predicted IC₅₀ value of the peptide against the predicted IC₅₀ values for a set of (on the order of 105) naturally occurring random peptides (see e.g., Jurtz V et al., J Immunol. 2017, which is incorporated herein by reference in its entirety). In some embodiments, if the predicted percentile rank≤2, a peptide is considered a binder. If it is ≤0.5, it is considered a strong binder.

At steps 112 and 114, computational mutagenesis can be performed in which, at step 112, the target peptide is mutated at a single amino acid position, resulting in several mutated peptides each having a single amino acid mutation from the target peptide. The mutated peptides are each associated with a respective peptide position (i.e., position of mutation). Preferably, the target peptide is mutated at every amino acid position, resulting in a number of mutated peptides equal to the number of amino acids in the target peptide. In some embodiments, mutated peptides can include a single amino acid mutation to glycine or an alanine amino acid. In some embodiments, the target peptide is mutated at single amino acid positions with the amino acid replaced by glycine, and positions of the target peptide having glycine are skipped, resulting in several mutated peptides corresponding to mutations of the target peptide at non-glycine positions.

At step 114, the binding affinity of each mutated peptide to the MHC molecule is predicted. In some embodiments, the binding affinity can be calculated using a computer-based model identical or similar to the computer-based model utilized at step 110.

At step 116, the binding affinity of the target peptide to the MHC molecule is compared to each binding affinity of the mutated peptides to the MHC molecule. The peptide positions associated with mutated peptides that do not result in the loss of binding affinity to the MHC molecule (compared to the target peptide binding affinity to the MHC molecule) are flagged. These flagged peptide positions are identified as not involved in binding to the MHC molecule and are therefore free to interact with an antigen-recognition molecule.

In some embodiments, when the binding affinity of a mutated peptide with a mutation associated with the respective position is approximately equal to the binding affinity of the target peptide to the MHC molecule, the respective position associated with the mutated peptide is predicted to be involved in interacting with an antigen-recognition molecule that recognizes the MHC-target peptide complex or otherwise flagged as free positions. Percentile rank values from NetMHCpan (e.g., NetMHCpan4.0 or NetMHCpan4.1) may be used. A mutated position is deemed as one resulting in loss of binding affinity to MHC molecule, if the percentile rank of the mutated peptide is less than a specific threshold, wherein the lower the percentile rank, the higher the binding affinity. Utilizing predicted percentile rank to quantify binding affinity, in some embodiments, if the rank of the target peptide is ≤0.5, then the threshold is 1.0. If the rank of the target peptide is >0.5 and ≤2.0, then the threshold is 2.0. If the rank of the target peptide is >2.0, then the threshold is 4.0. In other embodiments, a difference in binding affinity (e.g., percentile rank) between the mutated peptide and target peptide may be used to identify positions as involved in binding to the MHC molecule or free to interact with an antigen-recognition molecule. Positions where mutations result in a significant loss of binding affinity (e.g., a loss greater than a threshold) may be deemed to be involved in binding to the MHC molecule and positions where mutations do not result in a significant loss of binding affinity may be deemed to be free to interact with an antigen-recognition molecule. For example, if the rank of the target peptide is ≤0.5, then the difference in rank (loss) threshold may be 1.0. If the rank of the target peptide is >0.5 and ≤2.0, then the difference in rank threshold may be 1.5. If the rank of the target peptide is >2.0, then the difference in rank threshold may be 2.0.

At step 118, out of these flagged free positions, the ones that contain a non-glycine amino acid are identified as positions important for antigen-recognition molecule binding. In some embodiments, if a mutated position does not result in a significant loss of MHC molecule binding and the amino acid at the position is a non-glycine amino acid, then that position is considered important for antigen-recognition molecule binding. In embodiments in which, at step 112, the target peptide is mutated at single amino acid positions with the amino acid replaced by glycine and positions of the target peptide having glycine are skipped, step 118 can be omitted because all of the mutated peptides resulting from step 112, by definition, correspond to positions containing a non-glycine amino acid.

In some embodiments, when the structure of an MHC-target peptide complex bound to an antigen-recognition molecule is known, the binding motifs in the peptide sequence involved in antigen-recognition molecule interactions can be used as a comparison to verify that important positions for antigen-recognition molecule binding are correctly identified. Using immunopeptidomics data, predicted off-target peptides that have also been observed in mass-spectrometry experiments can provide additional indication that the predicted off-targets may be bona fide off-targets.

At step 106, similar peptides are identified. The similar peptides can be identified based on presence in an organism (e.g., human), and similarities to the target peptide at positions important for antigen-recognition molecule binding. Similar peptides may be selected such that they all be the same length as the target peptide. A similar peptide may be identical to the target peptide, although from a different protein source as the target peptide. However, in some instances, a target peptide may have already been screened such that non-unique candidate target peptides (candidate target peptides sharing an identical sequence with a peptide elsewhere in the proteome) are not selected as a target.

A working list of peptide sequences can be assembled to include peptide sequences in an organism (e.g., human). In some embodiments, the working list can be assembled based on canonical human protein sequences in a medical research database such as, as a non-limiting example, UniprotKB. A working list of peptide sequences can be filtered by comparing peptide sequences in the database to important positions of the target peptide identified in step 104 and keeping only peptide sequences having identical amino acids to the target peptide at a threshold number of important positions. The threshold number of positions can be two positions, three positions, four positions, or five positions. In embodiments in which a specific antigen-recognition molecule is not known, i.e. the PIGSPRED method 100 is agnostic to antigen-recognition molecule structure, three positions is preferred. While there may be an antigen-recognition molecule that binds at only two positions, such antigen-recognition molecule likely will have an impractically large number of off-target peptides. Increasing the threshold number of positions may result in underestimation of off-targets.

The working list of peptides can be further filtered to retain only peptides present in essential, normal tissues or essential cell types. Disrupting cell function in essential, normal tissues or essential cell types is more likely to cause more severe off-target effects, and therefore poses greater risk compared to disruption of cell function in non-essential tissues. In some embodiments, a medical research database can be utilized that includes gene expression in healthy donors. The Gene Tissue Expression database or GTEx is a non-limiting example of a suitable database, see gtexportal.org. For example, GTEx version 6 (v6), based on genome build GRCh38, contains gene expression data across 51 tissue types from 549 healthy donors. GTEx version 8 (v8), based on genome build GRCh38, contains gene expression data across 54 tissue types from 948 healthy donors; it is understood that additional donor and tissue samples are being sequenced for newer releases. In some embodiments, an alternative database to the GTEx database, and/or a different version of the GTEx database can be utilized to determine potential high risk off-target peptides in step 106, which may result in identifying additional or fewer potential high risk peptides, compared to specific examples disclosed herein, as understood by a person skilled in the pertinent art.

In some embodiments, gene expression values are queried in only those tissue types that are considered essential. Tissue types that may be considered non-essential may be those which may be sacrificed for the purpose of saving a patient's life, such as breast, ovary, testes, etc. In some embodiments, for each gene, the 95-percentile expression value in each essential tissue type is calculated, where 95-percentile expression value means 95% of all expression measurements of a gene in a tissue type falls at or below that value. Then, the maximum of the 95-percent expression value across all essential tissue types is calculated. If the maximum of the 95-percent expression value of a gene is greater than 0.5 transcripts per million (TPM) (which may be adjusted based on transcriptional noise), then it can be assumed that any similar peptide that is derived from the gene will be a potential high risk off-target peptide. High TPM means high expression and vice versa. To calculate TPM, divide read counts for a gene by the length of each gene in kilobases, resulting in reads per kilobase (RPK); next all RPK values in a sample are counted and divided by 1,000,000, resulting in a “per million” scaling factor; and finally divide each RPK value by the “per million” scaling factor, resulting in TPM for each gene. See, e.g., Li et al. (2010) Bioinformatics, 26 (4): 493-500; Varabyou et al. (2021) Genome Res. 31 (2): 301-308. The working list of peptide sequences can be filtered to remove peptides not identified as potential high risk. In some embodiments, an alternative database to the GTEx database, and/or a different version of the GTEx database can be utilized to determine potential high risk off-target peptides in step 106, which may result in identifying additional or fewer potential high risk peptides, compared to specific examples disclosed herein, as understood by a person skilled in the pertinent art.

In some embodiments, peptides that are filtered from the working list of peptide sequences at step 106 may be separately tracked as a list of potential secondary target peptides. A secondary target peptide may be derived from a secondary target gene that is distinct from the target gene of the target peptide, and cross-reactivity with the secondary target peptide may be therapeutically beneficial. If a potential secondary peptide is identical to the target peptide or highly similar, it is likely to cross-react with an antigen recognition molecule targeting the target peptide. Additional testing (e.g., antigen-recognition molecule screening) may optionally be performed on one or more of the potential secondary target peptides to assess whether cross-reactivity with those potential secondary target peptides could provide a beneficial therapeutic effect. Similar peptides derived from disease-associated genes or disease-associated antigens, such as cancer-specific genes or peptides that have been identified as or are derived from proteins that have been identified as cancer-specific or tumor-specific antigens, may exhibit beneficial cross-reactivity, and therefore may be a secondary target peptide. For example, cancer/testis (CT) antigens which are not expressed in normal, essential tissue may be tracked if similar to a target peptide, particularly where the target peptide is also a CT antigen, as exemplified in Example 1. Target peptides which share sequence identify (e.g., identical peptides or highly similar) with multiple suitable antigens for therapeutic targeting may be preferable.

At step 108, the working list of peptide sequences can be further filtered to remove peptides that do not bind to the MHC molecule and a degree of similarity (DOS) score can be calculated for remaining peptides in the working list. Binding affinity of peptides in the list of peptide sequences can be determined by utilizing a computer-based binding affinity model. Peptides that are not predicted to bind to the MHC molecule are removed from the working list of peptide sequences. In some embodiments, a peptide with percentile rank value≤2.0 is considered a binder. Optionally, a list of potential secondary target peptides, if being tracked, may likewise be filtered to remove peptides that do not bind to the MHC molecule in the same manner as described with respect to the working list of similar peptides in step 108. Also optionally, a degree of similarity (DoS) score can be calculated for potential secondary target peptides.

The DoS score can be based at least in part on number of amino acids of a respective peptide in the working list that is identical to amino acids at corresponding positions of the target peptide that are identified, at step 104, as important for binding to an antigen-recognition molecule. In some embodiments, the DoS score only considers those positions that are identified, at step 104, as important for binding to an antigen-recognition molecule. Alternatively, the DoS may consider at least some positions that are not identified, at step 104, as important for binding to an antigen-recognition molecule. For instance, in some embodiments, the DoS score can be based on the number of amino acids identified (at step 116) as not involved in binding to the MHC molecule, or the DoS score can be based on number of identical amino acids at every amino acid position of the respective peptide.

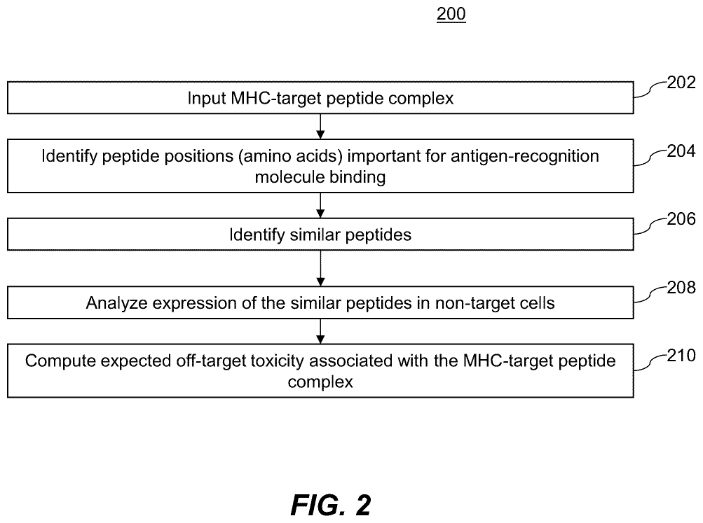

FIG. 2 illustrates a flow diagram of an embodiment of a method 200 to compute expected off-target toxicity associated with an MHC-target peptide complex. The method 200 is preferably executed by computational systems, engines, modules, devices, and/or networks. The method 200 is preferably stored as instructions on non-transitory computer-readable medium.

At step 202, an MHC-target peptide complex is provided as an input.

At step 204, peptide positions (amino acids) important for antigen-recognition molecule binding are identified. In preferred embodiments, the peptide positions are identified according to the method 104 illustrated in FIG. 1B. In some embodiments, the peptide positions are identified by analyzing the experimental structures of the MHC-target peptide complex with a specific antigen-recognition molecule.

At step 206, similar peptides can be identified. The similar peptides can be identified based on presence in an organism (e.g., human), similarities to the target peptide at positions important for antigen-recognition molecule binding, and ability to bind to the MHC molecule in the MHC-target peptide complex. Similar peptides may be selected such that they all are the same length as the target peptide. A similar peptide may be identical to the target peptide, although from a different protein source as the target peptide. However, in some instances, a target peptide may have already been screened such that non-unique candidate target peptides (candidate peptides sharing an identical sequence with a peptide elsewhere in the proteome) are not selected as a target.

A working list of peptide sequences can be assembled to include peptide sequences in an organism (e.g., human). The working list can be represented by an appropriate data structure such as a list, matrix, and/or other appropriate data structure as understood by a person skilled in the pertinent art. In some embodiments, the working list can be assembled based on canonical human protein sequences in a medical research database such as, as a non-limiting example, UniprotKB. In some embodiments, step 206 can include receiving the peptide sequences of an organism (e.g., human) as an input and utilizing the peptide sequences to generate the working list of peptide sequences.

The working list of peptide sequences can be filtered by comparing peptide sequences in the database to important positions of the target peptide identified in step 204 and keeping only peptide sequences having identical amino acids of the target peptide at or greater than a threshold number of important positions. The threshold number of positions can be two positions, three positions, four positions, or five positions. In embodiments in which a specific antigen-recognition molecule is not known, i.e. the PIGSPRED method 100 is agnostic to antigen-recognition molecule structure, three or four positions is preferred, and three positions is most preferred. The threshold number of positions is preferably determined based on the number of binding positions for an antigen-recognition molecule that is practical in a clinical setting. While there may be an antigen-recognition molecule that binds at only two positions, such antigen-recognition molecule likely will have an impractically large number of off-target peptides which may result in adverse side effects in a clinical setting, therefore such antigen-recognition molecule is impractical in a clinical setting. Increasing the threshold number of positions may result in underestimation of off-targets for antigen-recognition molecules that bind to fewer positions than the threshold number of positions; therefore the resulting expected off-target toxicity computed at step 210 may not be accurate for antigen-recognition molecules which bind to fewer than the threshold number of positions.

At step 208, expression of the similar peptides in non-target cells is analyzed. The risk associated with an antigen-recognition molecule binding to a respective peptide of the similar peptides identified in step 206 is greater if the respective peptide is present in essential, normal tissues or essential cell types. Disrupting cell function in essential, normal tissues or essential cell types is more likely to cause more severe off-target effects, and therefore poses greater risk compared to disruption of cell function in non-essential tissues. The working list of peptides can be further filtered to retain only peptides present in essential, normal tissues or essential cell types.

In some embodiments, a medical research database can be utilized that includes gene expression in healthy donors. The Gene Tissue Expression database or GTEx is a non-limiting example of a suitable database, see gtexportal.org. For example, GTEx version 6 (v6), based on genome build GRCh38, contains gene expression data across 51 tissue types from 549 healthy donors. GTEx version 8 (v8), based on genome build GRCh38, contains gene expression data across 54 tissue types from 948 healthy donors; it is understood that additional donor and tissue samples are being sequenced for newer releases. In some embodiments, gene expression values are queried in only those tissue types that are considered essential. Tissue types that may be considered non-essential may be those which may be sacrificed for the purpose of saving a patient's life, such as breast, ovary, testes, etc. In some embodiments, for each gene, the 95-percentile expression value in each essential tissue type is calculated and then the maximum of that value across all essential tissue types is calculated. If the maximum expression value of a gene is greater than 0.5 TPM (which may be adjusted based on transcriptional noise), then it can be assumed that any similar peptide that is derived from the gene will be a potential high risk off-target peptide. The working list of peptide sequences can be filtered to remove peptides not identified as potential high risk. However, in some embodiments, peptides not identified as potential high risk may not need to be discarded. As discussed above, such peptides may form “secondary target peptides.” Accordingly, another working list comprising the potential secondary target peptides may be prepared, and additional testing (e.g., antigen-recognition molecule screening) may optionally be performed on one or more potential secondary target peptides to assess whether cross-reactivity with those peptides could provide a beneficial therapeutic effect. In some embodiments, an alternative database to the GTEx database, and/or a different version of the GTEx database can be utilized to determine potential high risk off-target peptides in step 208, which may result in identifying additional or fewer potential high risk peptides, compared to specific examples disclosed herein, as understood by a person skilled in the pertinent art.

At step 210, expected off-target toxicity associated with the MHC-target peptide complex is computed. Once the potential high risk off-target peptides have been identified, a Degree of Similarity (DOS) score is calculated for each of the high risk off-target peptides. The DoS score quantifies the homology between the high risk off-target peptide and the target peptide. DoS represents the number of identical amino acids at identical positions or hamming distance between peptides. Using immunopeptidomics data, off-target peptides that have been observed in mass-spectrometry experiments can provide additional evidence for the potential off-targets.

The working list of peptide sequences can be filtered to remove peptides which have a DoS score below a given threshold. The number of peptide sequences remaining in the working list can be used as a metric indicating off-target toxicity of the MHC-target peptide complex which was input at step 202 to the method 200 illustrated in FIG. 2.

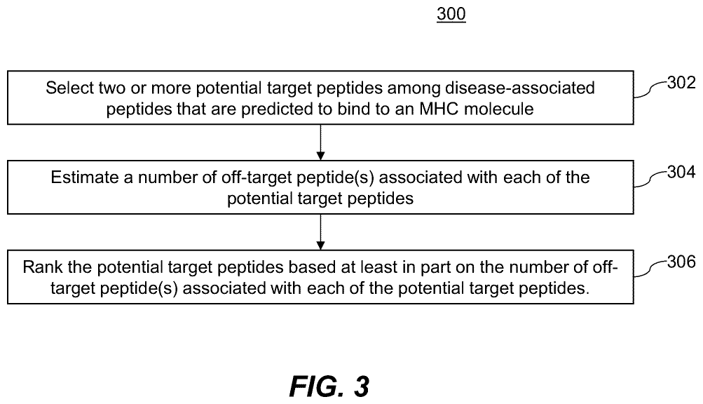

FIG. 3 illustrates a flow diagram of an embodiment of a method 300 for ranking potential target peptides to mitigate off-target toxicity.

At step 302, two or more potential peptides are selected among disease-associated peptides that are predicted to bind to an MHC molecule. Disease-specific MHC-target peptide complexes can be identified by ascertaining genes that are specifically expressed in diseased tissue. In some embodiments, the disease-specific MHC-target peptide complex can be identified based on a medical database such as The Cancer Genome Atlas (TCGA) and Genome Tissue Expression Database (GTEx), which include human gene sequences. In some embodiments, a gene that is expressed in a cancer type at 75-percentile TPM value >2 and is negligibly expressed in all essential, normal tissues or essential cell types in the GTEx is considered a cancer-specific gene. The canonical protein sequence corresponding to the cancer-specific gene can be derived from a medical research database including canonical human protein sequences such as the UniProtKB database.

The derived protein sequences can be used to predict the potential 8-25 mer peptide sequences that are predicted to bind the MHC of interest. When the MHC molecule is a class I MHC molecule, the predicted or detected peptides in step 302 can be 8-12 amino acids in length. When the MHC molecule is a class II MHC molecule, the predicted or detected peptides in step 302 can be 13-17 amino acids in length. The predictions can be done using a binding affinity calculation tool such as the NetMHCpan tool. In some embodiments, if the predicted binding affinity≤500 nM, a peptide is considered a binder. If it is ≤50 nM, it is considered a strong binder. In some embodiments, if the predicted percentile rank≤2, a peptide is considered a binder. If it is ≤0.5, it is considered a strong binder.

At step 304, a number of off-target peptide(s) associated with each of the potential target peptides can be estimated. In some embodiments, the number of off-target peptide(s) can be estimated utilizing steps of methods 100, 200 in FIGS. 1A and/or FIG. 2.

At step 306, the potential target peptides are ranked based at least in part on the number of off-target peptide(s) associated with each of the potential target peptides. In some embodiments, the number of potential off-targets estimated in step 304 is representative of the likelihood of off-target toxicity that might be associated with the MHC-target peptide complex, and thus this number can be used to rank the list of potential target peptides and to prioritize them for therapeutic development.

After the target selection and the generation of therapeutic molecules that bind the target, the off-targets predicted by PIGSPRED play an important role in experimental screening of therapeutic molecules for molecules that do not bind the off-targets. The most specific therapeutic molecules can be selected for further development.

In some embodiments, the method 300 illustrated in FIG. 3 can be executed by repeating the method 200 in FIG. 2, providing differing target MHC-target peptide complexes as input at step 202 and resulting in an off-target toxicity metric at step 210 for each of the input MHC-target peptide complexes. The off-target toxicity metrics can be compared to rank the MHC-target peptide complexes. The MHC-target peptide complex ranked as having lower off-target toxicity can be selected for further analysis, can be used for generation of antigen-recognition molecule(s), and/or can be used for testing of antigen-recognition molecule(s).

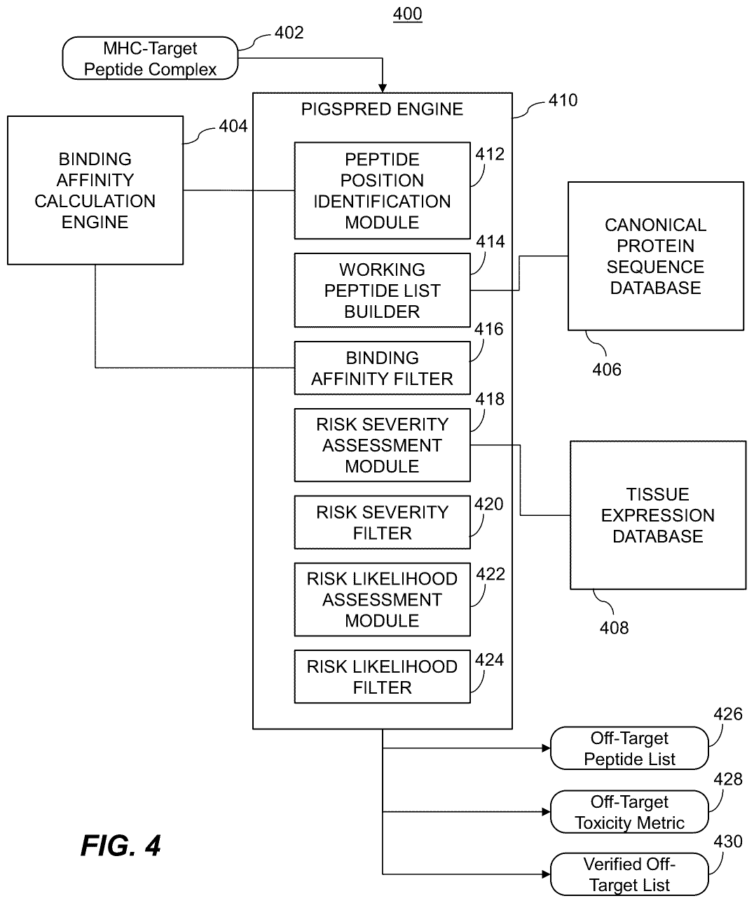

FIG. 4 illustrates a block diagram of an exemplary embodiment of a computational system 400 configured to output an off-target peptide list and/or an off-target toxicity metric. The system 400 includes an exemplary embodiment of a PIGSPRED engine 410. The PIGSPRED engine 410 is configured to receive, as an input, a computational representation of a target peptide presented in an MHC-target peptide complex 402. In the illustrated embodiment, the PIGSPRED engine 410 provides as an output an off-target peptide list 426 and/or an off-target toxicity metric 428.

The illustrated embodiment of the PIGSPRED engine 410 includes a peptide position identification module 412, a working peptide list builder 414, a binding affinity filter 416, a risk severity assessment module 418, a risk severity filter 420, a risk likelihood assessment module 422, and a risk likelihood filter 424. The PIGSPRED engine 410 includes one or more processor(s) and non-transitory computer-readable medium with instructions thereon, that when executed by the one or more processor(s) cause the PIGSPRED engine 410 to perform functions associated with each of the aforementioned features 412, 414, 416, 418, 420, 422, 424. In some embodiments, the PIGSPRED engine can be configured to provide as an output intermediate values determined by features 412, 414, 416, 418, 420, 422, 424 of the PIGSPRED engine 410. The features 412, 414, 416, 418, 420, 422, 424 are illustrated separately and in a specific order for illustrative purposes. As understood by a person skilled in the pertinent art, the features 412, 414, 416, 418, 420, 422, 424 can be combined, reordered, or otherwise implemented in numerous configurations to achieve the functionality disclosed in relation to the illustrated embodiment of the PIGSPRED engine 410.

The illustrated system 400 further includes a binding affinity calculation engine 404, a canonical protein sequence database 406, and a tissue expression database 408 in communication with the PIGSPRED engine 410. In a preferred embodiment, the binding affinity calculation engine 404, the canonical protein sequence database 406, and the tissue expression database 408 are separate from the PIGSPRED engine 410, and the PIGSPRED engine 410 is in communication with each. Alternatively, one or more of the ancillary features 404, 406, 408, 410 can be integral to the PIGSPRED engine 400 as understood by a person skilled in the pertinent art. The binding affinity calculation engine 404 includes a computer-based model for calculating binding affinity of a peptide to an MHC molecule. In some embodiments, the binding affinity calculation engine includes a commercial tool such as NetMHCpan or similar product. The canonical protein sequence database 406 includes information from which healthy tissue protein sequences can be derived. In some embodiments, the canonical protein sequence database 406 includes the UniProtKB database or similar medical database. As a non-limiting example, the tissue expression database 408 can include gene expression data across approximately 30 tissue types from approximately 549 healthy donors; in another non-limiting example, the tissue expression database 408 can include gene expression data across approximately 54 tissue types from approximately 948 healthy donors. In some embodiments, the tissue expression database 408 includes the Gene Tissue Expression database, GTEx, or similar medical database.

The peptide position identification module 412 of the PIGSPRED engine 410 can receive the computational representation of the MHC-target peptide complex 402 as an input and identify positions important for binding to an antigen-recognition molecule.

In a preferred embodiment, the peptide position identification module 412 is configured to perform the following steps: (a) receive, as an input, a computational representation of a target peptide in an MHC-target peptide complex; (b) determine binding affinity of the target peptide to the MHC molecule; (c) generate sequences of a plurality mutated peptides each associated with a mutation at a respective amino acid position of the target peptide; (d) determine binding affinity of each mutated peptide of the plurality of mutated peptides of the MHC molecule; (e) predict the amino acid position(s) involved in interacting with an antigen-recognition molecule that recognizes said MHC-target peptide complex based at least in part on a comparison of the binding affinity for each mutated peptide to the binding affinity of the target peptide; and (f) provide, as an output, an indication of the amino acid position(s) likely to be involved in interacting with the antigen-recognition molecule.

Steps (b) and (d) are executed by utilizing the binding affinity calculation engine 404. In some embodiments, at step (e) a threshold value of the binding affinity for a mutated peptide can be used to determine whether the amino acid position associate with the mutation is involved in interacting with an antigen-recognition molecule. In some embodiments, predicted percentile rank is utilized to quantify binding affinity, such that, if the rank of the target peptide is ≤0.5, then the threshold value of the binding affinity for the mutated peptide is 1.0; if the rank of the target peptide is >0.5 and ≤2.0, then the threshold value of the binding affinity for the mutated peptide is 2.0; and if the rank of the target peptide is >2.0, then the threshold value of the binding affinity for the mutated peptide is 4.0. A mutated position is deemed as one resulting in loss of binding affinity to MHC molecule. Percentile rank is inversely related to binding affinity, therefore when the threshold value of the binding affinity for a mutated peptide is based on percentile rank, a percentile rank less than the threshold value indicates the amino acid position associate with the mutation is involved in interacting with an antigen-recognition molecule.

The working list builder 414 is configured to generate a working list of peptides, within a total pool of predicted or detected peptides of suitable length, such that peptides listed in the working list each include at least two amino acids that (i) are located at positions corresponding to positions within the target peptide that are involved in interacting with the antigen-recognition molecule and (ii) are identical to the corresponding amino acids of the target peptide. In some embodiments, the working peptide list builder 414 can derive the total pool of predicted or detected peptides of suitable length from the canonical protein sequence database 406. When the MHC molecule is a class I MHC molecule, a peptide of suitable length may be 8-12 amino acids in length. A peptide of suitable length may be the same length as the target peptide. When the MHC molecule is a class II MHC molecule, a peptide of suitable length may be 13-17 amino acids in length. A peptide of suitable length may be the same length as the target peptide.

The binding affinity filter 414 is configured to determine binding affinity, of each of the peptides listed in the working list, to the MHC molecule and filter the working list to include only peptide(s) having a calculated binding affinity to the MHC molecule indicating a greater likelihood to bind compared a threshold value. The binding affinity filter 416 can utilize the binding affinity calculation engine 404 to determine the binding affinity of each of the peptides in the working list to the MHC molecule. In some embodiments, a peptide with percentile rank value ≤2.0 is considered a binder by the binding affinity filter 414, the percentile rank value being used as the threshold value. The threshold value used by the binding affinity filter 414 need not be equal to the threshold value used by the position identification module 412.

The risk severity assessment module 418 is configured to estimate the number of peptide(s) in the working list of off-target peptides which are expressed in essential, normal tissues or essential cell types. Disrupting cell function in essential, normal tissues or essential cell types is more likely to cause more severe off-target effects, and therefore poses greater risk compared to disruption of cell function in non-essential tissues. The risk severity assessment module 418 can utilize the tissue expression database 408 to determine which peptide(s) in the working list are expressed in essential, normal tissues or essential cell types. In some embodiments, gene expression values are queried in only those tissue types that are considered essential. Tissue types that may be considered non-essential may be those which may be sacrificed for the purpose of saving a patient's life, such as breast, ovary, testes, etc. In some embodiments, for each gene, the 95-percentile expression value in each essential tissue type is calculated and then the maximum of that value across all essential tissue types is calculated. If the maximum expression value of a gene is greater than 0.5 TPM (which may be adjusted based on transcriptional noise), then it can be assumed that any similar peptide that is derived from the gene will be a potential high risk off-target peptide. A similar peptide may be identical to the target peptide, although from a different protein source as the target peptide. However, in some instances, a target peptide may have already been screened such that non-unique candidate target peptides (candidate target peptides sharing an identical sequence with a peptide elsewhere in the proteome) are not selected as a target.

The risk severity filter 420 filters the working list to remove peptides not identified as potential high risk by the risk likelihood assessment module 418.

The risk likelihood assessment module 422 calculates a Degree of Similarity (DoS) score for the peptide(s) in the working list of peptides. The DoS score is based at least in part on a number of amino acids identical to amino acids at corresponding positions of the target peptide, which amino acids of the target peptide are involved in interacting with the antigen-recognition molecule. In some embodiments, the DoS score only considers those positions that are identified, by the peptide position identification module 412, as important for binding to an antigen-recognition molecule. Alternatively, the DoS may consider at least some positions that are not identified as important for binding to an antigen-recognition molecule. For instance, in some embodiments, the DoS score can be based on the number of amino acids not involved in binding to the MHC molecule, or the DoS score can be based on number of identical amino acids at every amino acid position of the respective peptide. A greater DoS score is utilized as a predictor of the likelihood that an antigen-recognition molecule which binds to the MHC-target peptide complex will also bind with a peptide in the working list of peptides. A greater likelihood of an off-target peptide binding to the antigen-recognition molecule therefore reflects a greater risk likelihood. In some embodiments, for off-target peptides for which immunopeptidomics data is available, off-target peptides that have been observed in mass-spectrometry experiments can provide additional evidence for the similarity of potential off-targets. For example, a similar peptide observed in the immunopeptidome (via mass-spectrometry) may be identified as an off-target peptide based on having a DoS score satisfying a DoS threshold that may be lower than a DoS threshold that would otherwise be used to identify a similar peptide as an off-target peptide when it has not been observed in the immunopeptidome (e.g., in some implementations a DoS score of 5 or greater may qualify a similar peptide as an off-target peptide if observed in the immunopeptidome, whereas a DoS score of 6 or greater may qualify a similar peptide that has not been observed in the immunopeptidome as an off-target peptide).

The risk likelihood filter 424 filters the working list to include only peptide(s) having a DoS score greater than a threshold value.

In the illustrated embodiment, the resulting working list represents a list of off-target peptides associated with the input MHC-target peptide complex 402. In the illustrated embodiment, the PIGSPRED engine 410 provides an off-target peptide list, which is the resulting working list, as an output.

In the illustrated embodiment, a total count of off-target peptides in the off-target peptide list 426 is utilized as an off-target toxicity metric 428, which can be provided as a second output to the PIGSPRED engine 410. In some embodiments, the off-target toxicity metric 428 can be based at least in part on other factors such as DoS scores of off-target peptides (e.g. average DoS score of all off-target peptides), a numerical quantification of how “essential” the tissues and/or cell types are that are associated with the off-target peptides, alignment of protein amino acid sequences, and/or a probability score related to off-target likelihood. The toxicity metric 428 can therefore be a mathematical function of one or more factors related to the off-target peptides and the potential risks associated with the off-target peptides as understood by a person skilled in the pertinent art informed by the teachings herein.

In the illustrated embodiment, the PIGSPRED engine further provides a list of verified off-target peptides 430 including peptides for which immunopeptidomics data is available and has been used to verify similarity of the off-target peptides to the target peptide.

In some embodiments, the PIGSPRED engine can be configured to output results of intermediate steps including, but not limited to binding affinity, DoS scores, the working list at various stages of being filtered.

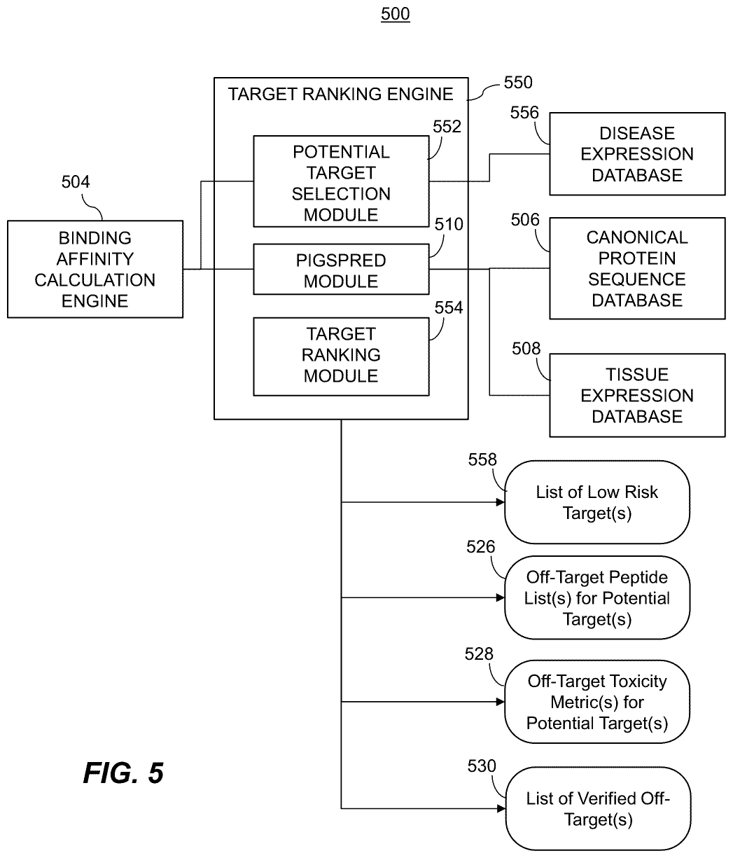

FIG. 5 illustrates a block diagram of an exemplary embodiment of a computations system 500 configured to output a list of low risk peptide targets 558, off-target peptide list(s) for potential target(s) 526, and/or off-target toxicity metric(s) for potential target(s) 528. The system 500 includes an exemplary embodiment of a target ranking engine 550.

The illustrated target ranking engine 550 includes a potential target selection module 552, a PIGSPRED module 510, and a target ranking module 554. The target ranking engine 550 includes one or more processor(s) and non-transitory computer-readable medium with instructions thereon, that when executed by the one or more processor(s) cause the target ranking engine 550 to perform functions associated with each of the aforementioned features 552, 510, 554. In some embodiments, the target ranking engine 550 can be configured to provide as an output intermediate values determined by features 552, 510, 554 of the target ranking engine 550. The features 552, 510, 554 are illustrated separately and in a specific order for illustrative purposes. As understood by a person skilled in the pertinent art, the features 552, 510, 554 can be combined, reordered, or otherwise implemented in numerous configurations to achieve the functionality disclosed in relation to the illustrated embodiment of the target ranking engine 550.

The illustrated system 500 further includes a binding affinity calculation engine 504, a disease expressing database 556, a canonical protein sequence database 506, and a tissue expression database 508. The binding affinity calculation engine 504, canonical protein sequence database 506, and tissue expression database 508 can be configured similarly to corresponding features 404, 406, 408 in FIG. 4. The disease expression database can include information to identify disease-specific MHC-target peptide complexes. In some examples, the disease expression database 556 can include TCGA database which include human gene sequences related to cancer. In a preferred embodiment, the binding affinity calculation engine 504, the disease expressing database 556, the canonical protein sequence database 506, and the tissue expression database 508 are separate from the target ranking engine 550, and the target ranking engine 550 is in communication with each. Alternatively, one or more of the ancillary features 504, 556, 506, 508 can be integral to the target ranking engine 550 as understood by a person skilled in the pertinent art.

The potential target selection module 552 is configured to be in communication with the disease expression database 556. The potential target selection module 552 is configured to determine a plurality of potential target peptides and associated MHC-target peptide complexes for each of the potential target peptides. The potential target selection module 552 can utilize the disease expression database 556 to identify potential target peptides based on type of disease under investigation, prevalence of the potential target peptide, etc. The potential target selection module 552 can determine binding affinity of the potential target peptides to one or more MHC molecules. The resulting MHC-target peptide complexes can be provided as input to the PIGSPRED module 510.

In the illustrated embodiment, the PIGSPRED module 510 functions similarly to the PIGSPRED engine 410 in FIG. 4, wherein MHC-target peptide complexes provided by the potential target selection module 552 each are provided as an input MHC-target peptide complex 402 to the system 400 in FIG. 4, and resulting off-target peptide list(s) 426, off-target toxicity metric(s) 428, and verified off-target peptide list 430 are provided as outputs for each MHC-target peptide complex respectively. In the illustrated embodiment, the off-target peptide list(s) for potential target(s) 526, off-target toxicity metric(s) for potential target(s) 528, and verified off-target peptide list(s) 530 are provided as outputs to the target ranking engine 550.

The target ranking module 554 is configured to rank the MHC-target peptide complexes according to risk. In some examples, the target ranking module 554 can provide a list of low risk target(s) 558 as an output to the target ranking engine 550.

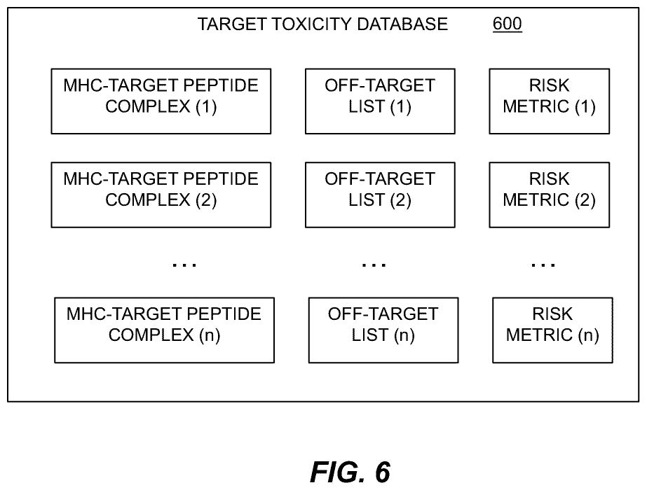

FIG. 6 illustrates a block diagram of an exemplary embodiment of a target toxicity database 600 including MHC-target peptide complexes, a list of their respective associated off-target peptides, and an associated risk metric. In a preferred embodiment, the off-target lists and risk metrics can be determined for each MHC-target peptide complex using the system 400 illustrated in FIG. 4. In a preferred embodiment, the MHC-target peptide complexes can be ranked using the system illustrated in FIG. 5.

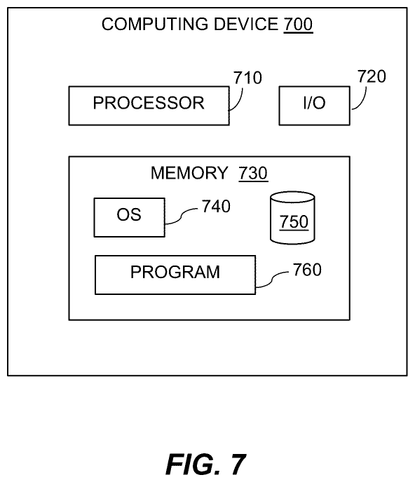

FIG. 7 illustrates a block diagram of an embodiment of a computing device 700. As shown, computing device 700 may include one or more processor(s) 710, an I/O device 720, a memory 730 containing an operating system (“OS”) 740, a database 750, and a program 760. In a preferred embodiment, the PIGSPRED engine 410 illustrated in FIG. 4 is realized by the computing device 700. In another preferred embodiment, the target ranking engine 550 can be realized by the computing device 700. In these preferred embodiments, instructions are stored in the memory 730 that are executable by the processor 710 to perform functions of the respective features 412, 414, 416, 418, 420, 422, 424, 552, 510, 554 of the respective engines 410, 510. In these preferred embodiments, the I/O device 720 is configured to communicate with the respective ancillary features 404, 406, 408, 504, 556, 506, 508 of the respective systems 400, 500 illustrated in FIGS. 4 and 5.

In some embodiments, the computing device 700 can include some or all of the features of the PIGSPRED engine 410 illustrated in FIG. 4 and/or the target ranking engine 550 illustrated in FIG. 5 and additional features, which can be realized as instructions in the memory 730. In one example embodiment, the instructions, when executed by the processor(s) 710, cause the computational device 700 to provide, as an input, a computational representation of the antigen-recognition molecule, the antigen-recognition molecule being capable of binding to the MHC-target peptide complex; determine binding affinity of the antigen-recognition molecule to a plurality of MHC-peptide complexes each including a respective likely off-target peptide from the working list and the MHC molecule; and filter the working list to include only likely off-target peptides for which the respective MHC-peptide complex has a binding affinity to the antigen-recognition molecule. The example embodiment may be utilized to screen specific therapeutic antigen-recognition molecules. Assuming there is a list of off-targets that is agnostic to the antigen-recognition molecule being used, this example embodiment finds off-targets for a specific antigen-recognition molecule by removing off-targets from the agnostic list that do not bind to the antigen-recognition molecule.

In another example embodiment, the instructions, when executed by the processor(s) 710, cause the computational device 700 to provide, as an input, off-target peptide expression in essential, normal tissues or essential cell types of a specific patient; and provide, as an output, an indication of the off-target effects for said patient. The example embodiment may be utilized to screen patients for clinical trial or other treatment.

Computing device 700 may be a single server or may be configured as a distributed computer system including multiple servers or computers that interoperate to perform one or more of the processes and functionalities associated with the disclosed embodiments. In some embodiments, computing device 700 may further include a peripheral interface, a transceiver, a mobile network interface in communication with processor 710, a bus configured to facilitate communication between the various components of computing device 700, and a power source configured to power one or more components of computing device 700. A peripheral interface may include the hardware, firmware and/or software that enables communication with various peripheral devices, such as media drives (e.g., magnetic disk, solid state, or optical disk drives), other processing devices, or any other input source used in connection with the instant techniques. In some embodiments, a peripheral interface may include a serial port, a parallel port, a general-purpose input and output (GPIO) port, a game port, a universal serial bus (USB), a micro-USB port, a high definition multimedia (HDMI) port, a video port, an audio port, a Bluetooth™ port, an NFC port, another like communication interface, or any combination thereof.

In some embodiments, a transceiver may be configured to communicate with compatible devices and ID tags when they are within a predetermined range. A transceiver may be compatible with one or more of: RFID, NFC, Bluetooth™, low-energy Bluetooth™ (BLE), WiFi™, ZigBee™, ABC protocols or similar technologies.

A mobile network interface may provide access to a cellular network, the Internet, or another wide-area network. In some embodiments, a mobile network interface may include hardware, firmware, and/or software that allows processor 710 to communicate with other devices via wired or wireless networks, whether local or wide area, private or public, as known in the art. A power source may be configured to provide an appropriate alternating current (AC) or direct current (DC) to power components.

Processor 710 may include one or more of a microprocessor, microcontroller, digital signal processor, co-processor or the like or combinations thereof capable of executing stored instructions and operating upon stored data. Memory 730 may include, in some implementations, one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like) for storing files, including an operating system, application programs (including, e.g., a web browser application, a widget or gadget engine, or other applications, as necessary), executable instructions, and data. In one embodiment, the processing techniques described herein are implemented as a combination of executable instructions and data within memory 730.

Processor 710 may be one or more known processing devices, such as a microprocessor from the Pentium™ family manufactured by Intel™ or the Turion™ family manufactured by AMD™. Processor 710 may constitute a single core or multiple core processor that executes parallel processes simultaneously. For example, processor 710 may be a single core processor that is configured with virtual processing technologies. In certain embodiments, processor 710 may use logical processors to simultaneously execute and control multiple processes. Processor 710 may implement virtual machine technologies, or other similar known technologies to provide the ability to execute, control, run, manipulate, store, etc. multiple software processes, applications, programs, etc. Other types of processor arrangements could be implemented that provide for the capabilities disclosed herein as understood by a person skilled in the pertinent art.

Computing device 700 may include one or more storage devices configured to store information used by processor 710 (or other components) to perform certain functions related to the disclosed embodiments. In one example, computing device 700 may include memory 730 that includes instructions to enable processor 710 to execute one or more applications, such as server applications, network communication processes, and any other type of application or software known to be available on computer systems. Alternatively, the instructions, application programs, etc., may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, computing device 700 may include memory 730 that includes instructions that, when executed by processor 710, perform one or more processes consistent with the functionalities disclosed herein. Methods, systems, and articles of manufacture consistent with disclosed embodiments are not limited to separate programs or computers configured to perform dedicated tasks. For example, computing device 700 may include memory 730 that may include one or more programs 760 to perform one or more functions of the disclosed embodiments. Moreover, processor 710 may execute one or more programs 760 located remotely from computing device 700. For example, computing device 700 may access one or more remote programs 760, that, when executed, perform functions related to disclosed embodiments.

Memory 730 may include one or more memory devices that store data and instructions used to perform one or more features of the disclosed embodiments. Memory 730 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational databases. Memory 730 may include software components that, when executed by processor 710, perform one or more processes consistent with the disclosed embodiments. In some embodiments, memory 730 may include database 750 for storing related data to enable computing device 700 to perform one or more of the processes and functionalities associated with the disclosed embodiments.

Computing device 700 may also be communicatively connected to one or more memory devices (e.g., databases (not shown)) locally or through a network. The remote memory devices may be configured to store information and may be accessed and/or managed by computing device 700. By way of example, the remote memory devices may be document management systems, Microsoft™ SQL database, SharePoint™ databases, Oracle™ databases, Sybase™ databases, or other relational databases. Systems and methods consistent with disclosed embodiments, however, are not limited to separate databases or even to the use of a database.

Computing device 700 may also include one or more I/O devices 720 that may include one or more interfaces for receiving signals or input from devices and providing signals or output to one or more devices that allow data to be received and/or transmitted by computing device 700. For example, computing device 700 may include interface components, which may provide interfaces to one or more input devices, such as one or more keyboards, mouse devices, touch screens, track pads, trackballs, scroll wheels, digital cameras, microphones, sensors, and the like, that enable computing device 700 to receive data from one or more users (such as via user device 130).

In example embodiments of the disclosed technology, computing device 700 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While computing device 700 has been described as one form for implementing the techniques described herein, other, functionally equivalent techniques may be employed as understood by a person skilled in the pertinent art. For example, as known in the art, some or all of the functionality implemented via executable instructions may also be implemented using firmware and/or hardware devices such as application specific integrated circuits (ASICs), programmable logic arrays, state machines, etc. Furthermore, other implementations may include a greater or lesser number of components than those illustrated.

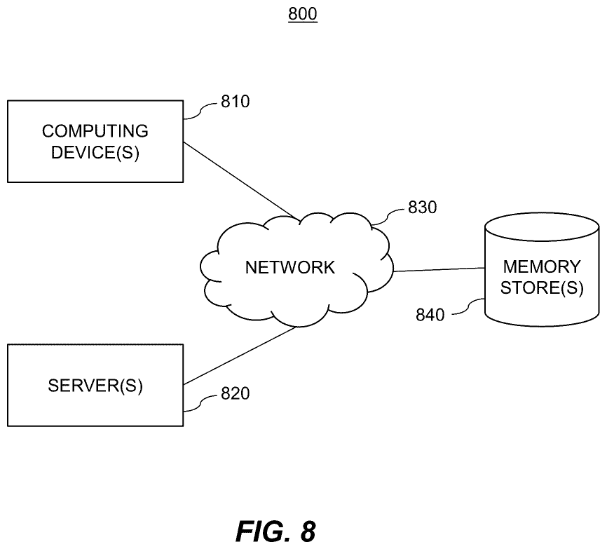

FIG. 8 illustrates a block diagram of an embodiment of a computing network 800 including computing device(s) 810, server(s) 820, memory store(s) 840, and a network 830 facilitating communication between each. In a preferred embodiment, computing device(s) 810 includes a computing device configured as illustrated in FIG. 7 and including the PIGSPRED engine 410 in FIG. 4 and/or the target ranking engine 550 in FIG. 5. In some embodiments, the computing device(s) 810 communicate with some or all of the ancillary features 404, 406, 408, 504, 556, 506, 508 via the network 830, wherein said ancillary features are hosted on the server(s) 820 and/or accessible from the memory store(s) 840.

Network 830 may be of any suitable type, including individual connections via the internet such as cellular or WiFi™ networks. In some embodiments, network 830 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

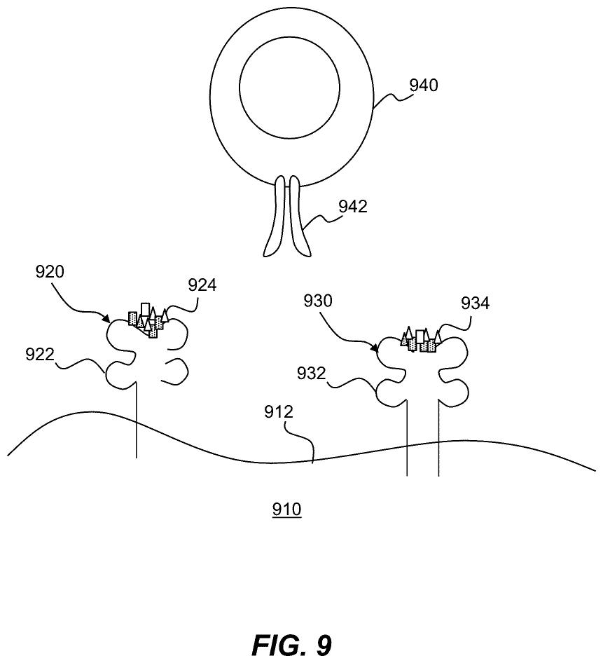

FIG. 9 illustrates cellular functions related to the example embodiments presented herein. A cell 910 includes MHC-peptide complexes 920, 930 presented on a surface 912 of the cell 910. The cell 910 includes a class I MHC-peptide complex 920 and a class II MHC-peptide complex 930. Each MHC-peptide complex 920, 930 includes a respective MHC molecule 922, 932 and a respective peptide 924, 934. Each peptide 924, 934 includes amino acids that are bound to the respective MHC molecule 922, 932 (illustrated as shaded shapes) and amino acids that are unbound to the respective MHC molecule 922, 932 (illustrated as white shapes). An antigen-recognition molecule 940 includes a receptor 942 that can bind to amino acids of a peptide in an MHC-target peptide complex that are unbound to the MHC molecule. The antigen-recognition molecule 940 may also bind to off-target peptides which have a similar configuration of amino acids (compared to the MHC-target peptide complex) that are unbound to the respective MHC molecule in an MHC-peptide complex.

Various methods described herein may relate to identifying (e.g., ranking/selecting) target peptides and/or to identifying (e.g., ranking/selecting) off-target peptides for a given target peptide. Any of these methods may further comprise synthesizing a target peptide and/or one or more off-target peptides identified. Methods for peptide synthesis include those known in the art and described herein. Any of these methods may further comprise loading a target peptide and/or one or more off-target peptides to an MHC molecule or any suitable component thereof to form a pMHC complex as described elsewhere herein. Any of these methods may further comprise binding a target peptide-MHC complex and/or one or more off-target peptide-MHC complexes to an antigen-recognition molecule (e.g., an antibody, TCR, or CAR). For example, any of these methods may comprise screening a target peptide-MHC complex and/or one or more off-target peptide MHC complexes for binding to an antigen-recognition molecule. Any of these methods may comprise incubating a target peptide and/or one or more off-target peptides with one or more cells (e.g., pulsing cells with peptide as described elsewhere herein).

In a further aspect, provided herein are off-target peptides identified using the methods described herein. Accordingly, the present disclosure also provides libraries comprising one or more of the off-target peptides identified using the methods described herein. In some embodiments, the libraries of the present disclosure may include the target peptides associated with the off-target peptides as well. In some embodiments, the libraries of the present disclosure may include off-target peptides identified by analyzing experimental structures of a pHLA in complex with an antigen-recognition molecule.

An exemplary peptide library of the present disclosure may comprise one or more off-target peptides identified for MAGEA4₂₃₀₋₂₃₉ target GVYDGREHTV (SEQ ID NO: 1) as described herein. In some embodiments, off-target peptides associated with MAGEA4₂₃₀₋₂₃₉ target GVYDGREHTV (SEQ ID NO: 1) include those listed in Tables 1B, 2B, and 3B herein. In some embodiments, an off-target peptide associated with MAGEA4₂₃₀₋₂₃₉ target GVYDGREHTV (SEQ ID NO: 1) comprises an amino acid sequence of any of SEQ ID NOs: 2-7, 9, 11-28, and 73-74 or a pharmaceutically acceptable salt thereof, or a fragment or derivative thereof. In some embodiments, an off-target peptide associated with MAGEA4₂₃₀₋₂₃₉ target GVYDGREHTV (SEQ ID NO: 1) consists essentially of an amino acid sequence of any of SEQ ID NOs: 2-7, 9, 11-28, and 73-74. In some embodiments, an off-target peptide associated with MAGEA4₂₃₀₋₂₃₉ target GVYDGREHTV (SEQ ID NO: 1) consists of an amino acid sequence of any one of SEQ ID NOs: 2-7, 9, 11-28, and 73-74.

Another exemplary peptide library of the present disclosure may comprise one or more off-target peptides identified for MAGEA4₂₈₆₋₂₉₄ target KVLEHVVRV (SEQ ID NO: 48) as described herein. In some embodiments, off-target peptides associated with MAGEA4₂₈₆₋₂₉₄ target KVLEHVVRV (SEQ ID NO: 48) include those listed in Table 4B herein. In some embodiments, an off-target peptide associated with MAGEA4₂₈₆₋₂₉₄ target KVLEHVVRV (SEQ ID NO: 48) comprises an amino acid sequence of any of SEQ ID NOs: 49-72 or a pharmaceutically acceptable salt thereof, or a fragment or derivative thereof. In some embodiments, an off-target peptide associated with MAGEA4₂₈₆₋₂₉₄ target KVLEHVVRV (SEQ ID NO: 48) consists essentially of an amino acid sequence of any of SEQ ID NOs: 49-72. In some embodiments, an off-target peptide associated with MAGEA4₂₈₆₋₂₉₄ target KVLEHVVRV (SEQ ID NO: 48) consists of an amino acid sequence of any one of SEQ ID NOs: 49-72.

Another exemplary peptide library of the present disclosure may comprise one or more off-target peptides identified for MAGEA3₁₆₈₋₁₇₆ target EVDPIGHLY (SEQ ID NO: 29) as described herein. In some embodiments, off-target peptides associated with MAGEA3₁₆₈₋₁₇₆ target EVDPIGHLY (SEQ ID NO: 29) include those listed in Table 5B herein. In some embodiments, an off-target peptide associated with MAGEA3₁₆₈₋₁₇₆ target EVDPIGHLY (SEQ ID NO: 29) comprises an amino acid sequence of any of SEQ ID NOs: 30-47 or a pharmaceutically acceptable salt thereof, or a fragment or derivative thereof. In some embodiments, an off-target peptide associated with MAGEA3₁₆₈₋₁₇₆ target EVDPIGHLY (SEQ ID NO: 29) consists essentially of an amino acid sequence of any of SEQ ID NOs: 30-47. In some embodiments, an off-target peptide associated with MAGEA3₁₆₈₋₁₇₆ target EVDPIGHLY (SEQ ID NO: 29) consists of an amino acid sequence of any one of SEQ ID NOs: 30-47.

A yet further exemplary peptide library of the present disclosure may comprise one or more peptides selected from: MAGEA4₂₃₀₋₂₃₉ target GVYDGREHTV (SEQ ID NO: 1), SEQ ID NOs: 2-7, 9, 11-28, and 73-74; MAGEA4₂₈₆₋₂₉₄ target KVLEHVVRV (SEQ ID NO: 48), SEQ ID NOs: 49-72; MAGEA3₁₆₈₋₁₇₆ target EVDPIGHLY (SEQ ID NO: 29), and SEQ ID NOs: 30-47, or any combination thereof.

Peptide libraries of present disclosure may comprise any number of a plurality of peptides as desired. For example, a peptide library of the present disclosure may comprise at least 2 peptides, at least 3 peptides, at least 4 peptides, at least 5 peptides, at least 6 peptides, at least 7 peptides, at least 8 peptides, at least 9 peptides, at least 10 peptides, at least 20 peptides, at least 30 peptides, at least 40 peptides, at least 50 peptides, or about 2-5 peptides, about 2-10 peptides, about 5-15 peptides, about 10-20 peptides, about 10-30 peptides, about 12-25 peptides, about 20-30 peptides, about 25-50 peptides, about 40-80 peptides, about 50-100 peptides, etc.

In a further aspect, provided herein are databases including computational representations of the off-target peptides identified using the methods described herein. The database can include information for each of the off-target peptides represented in any one of the exemplary libraries disclosed hereinabove. Accordingly, the databases may include computational representations of the target peptides associated with the off-target peptides as well. The computer-readable representations of the peptides in the database can include a peptide sequence or a computer model of the peptide. For instance, the database can include features of the database 600 illustrated in FIG. 6, including one or more lists of off-target peptides. The database 600 may or may not include the associated MHC-target peptide complex, and may or may not include the associated risk metric.

A peptide of the disclosure may be synthetically produced or produced by hydrolysis. Synthetically produced peptides can include randomly generated peptides, specifically designed peptides, and peptides where at least some of the amino acid positions are conserved among several peptides and the remaining positions are random. Alternatively, a peptide of the present disclosure may be produced by expression in a heterologous host cell.

In some embodiments, peptides of the disclosure can be synthesized by e.g., solid phase synthesis. As such, the peptides may be immobilized, for example to a solid support such as a bead. Peptides of the disclosure may be synthesized by the Fmoc-polyamide mode of solid-phase peptide synthesis. Temporary N-amino group protection is afforded by the 9-fluorenylmethyloxycarbonyl (Fmoc) group. Repetitive cleavage of this highly base-labile protecting group is done using 20% piperidine in N, N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives except for asparagine and glutamine, which are added using a reversed N, N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethanedithiol, phenol, anisole and water, the exact choice depending on the constituent amino acids of the peptide being synthesized. Also, a combination of solid phase and solution phase methodologies for the synthesis of peptides is possible.

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers.

Purification may be performed by techniques such as re-crystallization, ion-exchange chromatography, size exclusion chromatography, hydrophobic interaction chromatography and reverse-phase high performance liquid chromatography using e.g. acetonitrile/water gradient separation, or a combination thereof.

Peptides may be analyzed using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

Alternatively, the peptide may be produced by recombinant expression in a heterologous host cell. Such methods typically involve the use of a vector comprising a nucleic acid sequence encoding the peptide to be expressed, to express the polypeptide in vivo; for example, in bacteria, yeast, insect or mammalian cells.

In further embodiments, in vitro cell-free systems may be used. The peptides may be isolated and/or may be provided in substantially pure form. For example, they may be provided in a form which is substantially free of other peptides or proteins.

In some embodiments, the peptides of the disclosure may be about 8-25 amino acids in length. In some embodiments, the peptides of the disclosure may be about 8-12 amino acids in length. For example, a peptide disclosed herein may be 8 amino acids, 9 amino acids, 10 amino acids, 11 amino acids, or 12 amino acids in length. In some embodiments, the peptides of the disclosure may be about 13-17 amino acids in length. For example, a peptide disclosed herein may be 13 amino acids, 14 amino acids, 15 amino acids, 16 amino acids, or 17 amino acids in length.

The peptides of the disclosure may comprise one or more chemical modifications. Non-limiting examples of chemical modifications include, for example, phosphorylation, acetylation, deamidation acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulfides with other thiol compounds, reaction with maleimide, carboxymethylation with iodoacetic acid or iodoacetamide and carbamoylation with cyanate at alkaline pH. Chemical modifications may not correspond to those that may be present in vivo.

For example, modification of, for example, arginyl residues in proteins may be based on the reaction of vicinal dicarbonyl compounds such as phenylglyoxal, 2,3-butanedione, and 1,2-cyclohexanedione to form an adduct. Another example is the reaction of methylglyoxal with arginine residues. Cysteine can be modified without concomitant modification of other nucleophilic sites such as lysine and histidine. Selective reduction of disulfide bonds in proteins can also be performed. Disulfide bonds can be formed and oxidized during the heat treatment of biopharmaceuticals. Woodward's Reagent K may be used to modify specific glutamic acid residues. N-(3-(dimethylamino) propyl)-N′-ethylcarbodiimide can be used to form intra-molecular crosslinks between a lysine residue and a glutamic acid residue. For example, diethylpyrocarbonate and 4-hydroxy-2-nonenal can be used to modify histidyl residues in proteins. The reaction of lysine residues and other α-amino groups is, for example, useful in binding of peptides to surfaces or the cross-linking of proteins/peptides. Lysine is the site of attachment of poly (ethylene) glycol and the major site of modification in the glycosylation of proteins. Methionine residues in proteins can be modified with e.g. iodoacetamide, bromoethylamine, and chloramine T. Tetranitromethane and N-acetylimidazole can be used for the modification of tyrosyl residues. Cross-linking via the formation of dityrosine can be accomplished with hydrogen peroxide/copper ions. N-bromosuccinimide, 2-hydroxy-5-nitrobenzyl bromide or 3-bromo-3-methyl-2-(2-nitrophenylmercapto)-3H-indole (BPNS-skatole) have been used in recent studies for the modification of tryptophan.

Peptides described herein may comprise one or more (e.g., 1, 2, 3, or 4) amino acid substitutions and/or insertions and/or deletions. Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue. One or more (e.g., 1, 2, 3 or 4) amino acids may be substituted and/or inserted and/or deleted from the sequence of any one of SEQ ID NOs: 1-7, 9, and 11-74. Each substitution and/or insertion and/or deletion can take place at any position of any one of SEQ ID NOs: 1-7, 9, and 11-74.

In some embodiments, the peptides of the disclosure may comprise additional amino acids (e.g., 1, 2, 3 or 4) at the C-terminal end and/or at the N-terminal end of the sequence of any one SEQ ID NOs: 1-7, 9, and 11-74. A peptide of the disclosure may comprise the amino acid sequence of any one of SEQ ID NOs: 1-7, 9, and 11-74 except for one or more (e.g., 1, 2, 3, or 4) amino acid substitutions, insertions or deletions.

Amino acid substitutions may be conservative, by which it is meant the substituted amino acid has similar chemical properties to the original amino acid. For example, the following groups of amino acids share similar chemical properties such as size, charge, and polarity: Group 1-Ala, Ser, Thr, Pro, Gly; Group 2-Asp, Asn, Glu, Gln; Group 3-His, Arg, Lys; Group 4-Met, Leu, Ile, Val, Cys; Group 5-Phe, Thy, Trp.

In another aspect, the disclosure provides a complex of a peptide of the disclosure and an MHC molecule (pMHC complex). Preferably, the peptide is bound to the peptide binding groove of the MHC molecule. In some embodiments, the peptide and the MHC molecule form a non-covalent complex. In other embodiments, the peptide and the MHC molecule may be covalently linked, for example, via a linker. Accordingly, the present disclosure also provides libraries comprising one or more of the pMHC complexes described herein.

An exemplary pMHC complex library of the present disclosure may comprise one or more pMHC complexes comprising an off-target peptide associated with MAGEA4₂₃₀₋₂₃₉ target GVYDGREHTV (SEQ ID NO: 1) as described herein. In some embodiments, off-target peptides associated with MAGEA4₂₃₀₋₂₃₉ target GVYDGREHTV (SEQ ID NO: 1) present in the pMHC complexes include those listed in Tables 1B, 2B, and 3B herein. In some embodiments, an off-target peptide associated with MAGEA4₂₃₀₋₂₃₉ target GVYDGREHTV (SEQ ID NO: 1) present in a pMHC complex comprises an amino acid sequence of any of SEQ ID NOs: 2-7, 9, 11-28, and 73-74 or a pharmaceutically acceptable salt thereof, or a fragment or derivative thereof. In some embodiments, an off-target peptide associated with MAGEA4₂₃₀₋₂₃₉ target GVYDGREHTV (SEQ ID NO: 1) present in a pMHC complex consists essentially of an amino acid sequence of any of SEQ ID NOs: 2-7, 9, 11-28, and 73-74. In some embodiments, an off-target peptide associated with MAGEA4₂₃₀₋₂₃₉ target GVYDGREHTV (SEQ ID NO: 1) present in a pMHC complex consists of an amino acid sequence of any one of SEQ ID NOs: 2-7, 9, 11-28, and 73-74.

Another exemplary pMHC complex library of the present disclosure may comprise one or more pMHC complexes comprising an off-target peptide associated with MAGEA4₂₈₆₋₂₉₄ target KVLEHVVRV (SEQ ID NO: 48) as described herein. In some embodiments, off-target peptides associated with MAGEA4₂₈₆₋₂₉₄ target KVLEHVVRV (SEQ ID NO: 48) present in pMHC complexes include those listed in Table 4B herein. In some embodiments, an off-target peptide associated with MAGEA4₂₈₆₋₂₉₄ target KVLEHVVRV (SEQ ID NO: 48) present in a pMHC complex comprises an amino acid sequence of any of SEQ ID NOs: 49-72 or a pharmaceutically acceptable salt thereof, or a fragment or derivative thereof. In some embodiments, an off-target peptide associated with MAGEA4₂₈₆₋₂₉₄ target KVLEHVVRV (SEQ ID NO: 48) present in a pMHC complex consists essentially of an amino acid sequence of any of SEQ ID NOs: 49-72. In some embodiments, an off-target peptide associated with MAGEA4₂₈₆₋₂₉₄ target KVLEHVVRV (SEQ ID NO: 48) present in a pMHC complex consists of an amino acid sequence of any one of SEQ ID NOs: 49-72.

Another exemplary pMHC complex library of the present disclosure may comprise one or more pMHC complexes comprising an off-target peptide associated with MAGEA3₁₆₈₋₁₇₆ target EVDPIGHLY (SEQ ID NO: 29) as described herein. In some embodiments, off-target peptides associated with MAGEA3₁₆₈₋₁₇₆ target EVDPIGHLY (SEQ ID NO: 29) include those listed in Table 5B herein. In some embodiments, an off-target peptide associated with MAGEA3₁₆₈₋₁₇₆ target EVDPIGHLY (SEQ ID NO: 29) present in pMHC complexes comprises an amino acid sequence of any of SEQ ID NOs: 30-47 or a pharmaceutically acceptable salt thereof, or a fragment or derivative thereof. In some embodiments, an off-target peptide associated with MAGEA3₁₆₈₋₁₇₆ target EVDPIGHLY (SEQ ID NO: 29) present in a pMHC complex consists essentially of an amino acid sequence of any of SEQ ID NOs: 30-47. In some embodiments, an off-target peptide associated with MAGEA3₁₆₈₋₁₇₆ target EVDPIGHLY (SEQ ID NO: 29) present in a pMHC complex consists of an amino acid sequence of any one of SEQ ID NOs: 30-47.

A yet further exemplary pMHC complex library of the present disclosure may comprise one or more pMHC complexes comprising a peptide selected from: MAGEA4₂₃₀₋₂₃₉ target GVYDGREHTV (SEQ ID NO: 1), SEQ ID NOs: 2-7, 9, 11-28, and 73-74; MAGEA4₂₈₆₋₂₉₄ target KVLEHVVRV (SEQ ID NO: 48), SEQ ID NOs: 49-72; MAGEA3₁₆₈₋₁₇₆ target EVDPIGHLY (SEQ ID NO: 29), and SEQ ID NOs: 30-47, or any combination thereof.

pMHC complex libraries of present disclosure may comprise any number of a plurality of pMHC complexes as desired. For example, a pMHC complex library of the present disclosure may comprise at least 2 pMHC complexes, at least 3 pMHC complexes, at least 4 pMHC complexes, at least 5 pMHC complexes, at least 6 pMHC complexes, at least 7 pMHC complexes, at least 8 pMHC complexes, at least 9 pMHC complexes, at least 10 pMHC complexes, at least 20 pMHC complexes, at least 30 pMHC complexes, at least 40 pMHC complexes, at least 50 pMHC complexes, or about 2-5 pMHC complexes, about 2-10 pMHC complexes, about 5-15 pMHC complexes, about 10-20 pMHC complexes, about 10-30 pMHC complexes, about 12-25 pMHC complexes, about 20-30 pMHC complexes, about 25-50 pMHC complexes, about 40-80 pMHC complexes, about 50-100 pMHC complexes, etc.

MHC molecules used in pMHC complexes described herein include naturally occurring full-length MHC molecules as well as individual chains of MHC molecules (e.g., MHC class I α (heavy) chain, β2-microglobulin, MHC class II α chain, and MHC class II β chain), individual subunits of such chains of MHCs (e.g., α1, α2 and/or α3 subunits of MHC class I α chain, α1 and/or α2 subunits of MHC class II α chain, β1 and/or β2 subunits of MHC class II B chain) as well as fragments, mutants, and various derivatives thereof (including fusion proteins, e.g., fusions with viral envelope proteins or fusogens), wherein such fragments, mutants, and derivatives retain the ability to display an antigenic determinant for recognition by an antigen-recognition molecule.

Naturally-occurring MHC molecules are encoded by a cluster of genes on human chromosome 6 or mouse chromosome 17. MHCs are also referred to as H-2 in mice and Human Leucocyte Antigen (HLA) in humans. MHC class I molecules specifically bind CD8 molecules expressed on cytotoxic T lymphocytes (CD8+ T cells), whereas MHC class II molecules specifically bind CD4 molecules expressed on helper T lymphocytes (CD4+ T cells). MHCs include, but are not limited to, HLA specificities such as A (e.g., A1-A74), B (e.g., B1-B77), C (e.g., C1-C11), D (e.g., DI-D26), E, G, DR (e.g., DR1-DR8), DQ (e.g., DQ1-DQ9) and DP (e.g., DP1-DP6). More preferably, HLA specificities include A1, A2, A3, A11, A23, A24, A28, A30, A33, β7, β8, β35, β44, β53, β60, β62, DR1, DR2, DR3, DR4, DR7, DR8, and DR-11.

In some embodiments, the MHC molecule in a pMHC complex of the present disclosure is a human leukocyte antigen (HLA) molecule. The MHC molecule may be a human HLA molecule selected from the group consisting of HLA-A, HLA-B, HLA-C, HLA-E, HLA-F, and HLA-G. In some embodiments, the MHC class I or MHC II polypeptides may be derived from any functional human HLA-A, B, C, DR, or DQ molecules. Non-limiting examples of HLA-A alleles comprise, without limitation, A*01:01, A*02:01, A*02:02, A*03:01, A*11:01, A*23:01, A*24:02, A*25:01, A*26:01, A*29:01, A*29:02, A*31:01, A*32:01, A*33:01, A*34:01, A*36:01, A*43:01, A*66:01, A*68:01, A*69:01, A*74:01, and A*80:01. Non-limiting examples of HLA-B alleles comprise, without limitation, B*07:02. B*08:01, B*13:01, B*14:01, B*14:02, B*15:01, B*18:01, B*18:02, B*27:01, B*27:02, B*35:01, B*35:02, B*37:01, B*38:01, B*39:01, B*40:01, B*41:01, B*42:01, B*44:02, B*45:01, B*46:01, B*47:01, B*48:01, B*49:01, B*50:01, B*51:01, B*52:01, B*53:01, B*54:01, B*55:01, B*55:02, B*56:01, B*57:01, B*58:01, B*59:01, B*67:01, B*73:01, B*15:17, B*81:01, B*82:01, and B*83:01. Non-limiting examples of HLA-C alleles comprise, without limitation, Cw*01:01, Cw*02:02, Cw*03:03, Cw*04:01, Cw*05:01, Cw*06:02, Cw*07:01, Cw*07:02, Cw*08:02, Cw*12:03, Cw*14:01, Cw*15:02, Cw*16:01, Cw*17:01, and. Cw*18:01. Non-limiting examples of HLA-DR alleles comprise, without limitation, DRB1*01:01, DRB1*01:03, DRB1*15:01, DRB1*15:02, DRB1*16:01, DRB1*16:02, DRB1*03:01, DRB1*04:01, DRB1*04:04, DRB1*11:01, DRB1*12:01, DRB1*13:01, DRB1*13:02, DRB1*14:01, DRB1*14:02, DRB1*07:01, DRB1*08:01, DRB1*08:02, DRB1*08:03, DRB1*09:01, and DRB1*10:01.

In some embodiments, the MHC class I molecule may be selected from HLA-A*02, HLA-A*01, HLA-A*03, HLA-A*11, HLA-A*23, HLA-A*24, HLA-B*07, HLA-B*08, HLA-B*40, HLA-B*44, HLA-B*15, HLA-C*04, HLA*C*03, and HLA-C*07. There are also allelic variants of the above HLA types, all of which are encompassed by the present disclosure.

In some embodiments, the MHC molecule may be HLA-A*02:01 or HLA-A*01:01.

Naturally occurring MHC class I molecules consist of an a (heavy) chain associated with β2-microglobulin. The heavy chain consists of subunits α1-α3. The β2-microglobulin protein and α3 subunit of the heavy chain are associated. In certain embodiments, β2-microglobulin and α3 subunit are covalently bound. In certain embodiments, β2-microglobulin and α3 subunit are non-covalently bound. The α1 and α2 subunits of the heavy chain fold to form a groove for a peptide to be displayed and recognized by TCR.

In some embodiments, the MHC contained in a pMHC complex of the disclosure comprises (i) a class I MHC polypeptide or a fragment, mutant or derivative thereof, and, optionally, (ii) a β2 microglobulin polypeptide or a fragment, mutant or derivative thereof. In one specific embodiment, the class I MHC polypeptide is linked to the β2 microglobulin polypeptide by a peptide linker.

pMHC complexes of the disclosure may be isolated and/or in a substantially pure form. For example, the complex may be provided in a form which is substantially free of other peptides or proteins. MHC molecules as disclosed herein can include recombinant MHC molecules, non-naturally occurring MHC molecules, and functionally equivalent fragments of MHC, including derivatives or variants thereof, provided that peptide binding is retained. For example, MHC molecules may be attached to a solid support, in soluble form, attached to a tag, biotinylated and/or in multimeric form. A peptide disclosed herein may be covalently attached to the MHC.

Methods to produce soluble recombinant MHC molecules with which peptides disclosed herein can form a complex include, but are not limited to, expression and purification from E. coli cells or insect cells. Alternatively, MHC molecules may be produced synthetically, or using cell free systems.

The peptides disclosed herein may be presented on the surface of a cell in complex with MHC. Thus, the present disclosure also provides a cell presenting on its surface a pMHC complex disclosed herein. Such a cell may be a mammalian cell, preferably a cell of the immune system, and a specialized antigen-presenting cell (APC) such as a dendritic cell or a B cell. Other preferred cells include T2 cells. Cells presenting a peptide or pMHC complex of the disclosure may be isolated, preferably in the form of a homogenous population, or provided in a substantially pure form. Such cells may not naturally present a pMHC complex of the disclosure, or alternatively said cells may present the pMHC complex at a level higher than they would in nature. Such cells may be obtained by pulsing said cells with one or more peptides (e.g., 2 to 10, 2 to 20, 2 to 30, 5 to 25, 5 to 20, or 10 to 15 peptides) of the disclosure, or genetically modifying the cells (via DNA or RNA transfer) to express one or more peptides (e.g., 2 to 10, 2 to 20, 2 to 30, 5 to 25, 5 to 20, or 10 to 15 peptides) of the disclosure. Pulsing involves incubating the cells with the peptide for several hours using peptide concentrations typically ranging from 10⁻⁵ to 10⁻¹² M. Such cells may additionally be transduced with HLA molecules, such as HLA-A*02 to further induce presentation of the peptide(s). Cells may be produced recombinantly. Cells presenting peptides of the disclosure may be used to isolate antigen-binding molecules (e.g., antibodies, T cells, TCRs and CARs) which can bind to the cells.

Peptides or pMHC complexes disclosed herein may be fused or conjugated to one or more heterologous molecules. Peptides or pMHC complexes of the disclosed herein may also be in multimeric form. Accordingly, the present disclosure also provides fusion proteins, conjugates, and oligomeric complexes comprising a peptide or a pMHC complex of the disclosure.

In some embodiments, peptides are fused or conjugated to one or more heterologous molecules which can include an MHC molecule (or fragments thereof).

Heterologous molecules suitable for genetical fusion and/or chemical conjugation with the peptides or the pMHC complexes of the disclosure include, but are not limited to, peptides, polypeptides, small molecules, polymers, nucleic acids, lipids, sugars, etc. The heterologous molecule(s) may be fused at the N- and/or C-terminus of the peptide and/or another polypeptide chain in the pMHC complex.

Heterologous peptides and polypeptides include, but are not limited to, an epitope (e.g., FLAG) or a tag sequence (e.g., His₆, and the like) to allow for the detection and/or isolation of a fusion protein; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half-life-extending sequence comprising a combination of two or more (e.g., 2, 5, 10, 15, 20, 25, etc.) naturally occurring or non-naturally occurring charged and/or uncharged amino acids (e.g., Ser, Gly, Glu or Asp) designed to form a predominantly hydrophilic or predominantly hydrophobic fusion partner for a fusion protein; a functional or non-functional antibody (e.g., an antibody that is specific for dendritic cells), or a heavy or light chain thereof; and a polypeptide which has an activity different from fusion proteins of the present disclosure.

In some embodiments, fusion proteins of the disclosure may comprise one or more affinity tags, e.g., to allow for affinity purification or coupling to another molecule. Examples of affinity tags include, but are not limited to, a His₆ tag, an Avi-tag, a biotin, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, and Ubiquitin tag.

Peptides or pMHC complexes of the disclosure may be provided in soluble form, or may be immobilized by attachment to a suitable solid support. Examples of solid supports include, but are not limited to, a bead, a membrane, sepharose, a magnetic bead, a plate, a tube, a column. pMHC complexes may be attached to an ELISA plate, a magnetic bead, or a surface plasmon resonance biosensor chip. Methods of attaching peptides or pMHC complexes to a solid support are known to the skilled person, and include, for example, using an affinity binding pair, e.g. biotin and streptavidin, or antibodies and antigens. In some embodiments, peptides or pMHC complexes are labeled with biotin and attached to streptavidin-coated surfaces.

In another aspect, the disclosure provides an isolated polynucleotide comprising a nucleic acid sequence encoding one or more peptide(s) and/or peptide-based molecules (such as complexes (e.g., pMHC complexes), fusion proteins, or conjugates comprising the described peptides) of the disclosure. The polynucleotide may be, for example, DNA, cDNA, PNA, RNA or combinations thereof, either single- and/or double-stranded, or native or stabilized forms of polynucleotides, such as, for example, polynucleotides with a phosphorothioate backbone and it may or may not contain introns so long as it codes for the peptide.

In some embodiments, a polynucleotide described herein encodes a peptide comprising an amino acid sequence of any one of SEQ ID NOs: 1-7, 9, and 11-74, or a fragment or derivative thereof. In some embodiments, the polynucleotide described herein encodes a peptide comprising an amino acid sequence of any one of SEQ ID NOs: 1-7, 9, and 11-74, or a fragment or derivative thereof.

In a further aspect, the disclosure provides a vector comprising a nucleic acid sequence of the disclosure. The vector may include, in addition to a nucleic acid sequence encoding only a peptide of the disclosure, one or more additional nucleic acid sequences encoding one or more additional peptides. Such additional peptides may, once expressed, be fused to the N-terminus or the C-terminus of the peptide of the disclosure. Examples of such additional peptides are detailed in the sections above. In one embodiment, the vector includes a nucleic acid sequence encoding a peptide or protein tag such as, for example, a biotinylation site, a FLAG-tag, a MYC-tag, an HA-tag, a GST-tag, a Strep-tag or a poly-histidine tag.

The off-target peptides identified herein may be used for evaluation and/or screening of therapeutic molecules for molecules that have minimal off-target effects. Such therapeutic molecules can include antigen-recognition molecules such as T cell Receptors (TCRs), chimeric antigen receptors (CARs), antibodies, or antigen-binding fragments thereof. In various embodiments of the present disclosure, the antigen-recognition molecule is present in a solution. In various embodiments of the present disclosure, the antigen-recognition molecule is present on a cell (e.g., T cell, B cell or hybridoma).

In one aspect, the disclosure provides an in vitro method of assessing off-target effects of an antigen-recognition molecule, said method comprising (a) contacting the antigen-recognition molecule with a target peptide presented in a complex with a major histocompatibility complex (MHC) molecule (MHC-target peptide complex); (b) contacting the antigen-recognition molecule with one or more off-target peptides associated with said target peptide, wherein each of said off-target peptides is presented in a complex with the same MHC molecule as in (a) (MHC-off-target peptide complex); and (c) determining and comparing the level of binding of the antigen-recognition molecule to MHC-target peptide complex and each of the MHC-off-target peptide complexes.

In one aspect, the disclosure provides an in vitro method of assessing off-target effects of an antigen-recognition molecule, comprising a) contacting the antigen-recognition molecule with one or more off-target peptides associated with a target peptide that is recognized by the antigen-recognition molecule, wherein each of said off-target peptides is presented in a complex with a major histocompatibility complex (MHC) molecule (MHC-off-target peptide complex); and b) determining the level of binding of the antigen-recognition molecule to each of the MHC-off-target peptide complexes.

In some embodiments of the in vitro methods described above, the level of binding is determined by detecting the amount of antigen-recognition molecules bound to the MHC-peptide complexes. For example, if a signal correlated to binding of an antigen-recognition molecule to an MHC peptide complex has a S: N ratio (such as that displayed in Example 2, Table 7) of at least about 2.0, 2.5, 3.0, 3.5, 4.0, or 5.0 then the antigen-recognition molecule may be determined to exhibit detectable binding to the MHC peptide complex. If an antigen-recognition molecule specifically binds to one or more MHC-off-target peptide complexes, then the antigen-recognition molecule may exhibit off-target effects. In some specific embodiments, a S: N ratio of at least about 3 is indicative of detectable binding. In some embodiments, binding affinity may be determined. Binding affinity may be determined by measuring an equilibrium dissociation constant (KD) of the binding reaction. Alternatively, binding affinity may be characterized using other methods.

In some embodiments, the in vitro methods include determining that the antigen-recognition molecule is likely to have off-target effects if it detectably binds to at least one MHC-off-target peptide complex, wherein the off-target peptide is expressed in essential, normal tissues.

In another aspect, the disclosure provides a method of selecting an antigen-recognition molecule that binds a target pMHC complex with minimal off-target effects. Such method can comprise (a) contacting a plurality of antigen-recognition molecules with a target peptide presented in a complex with a major histocompatibility complex (MHC) molecule (MHC-target peptide complex); (b) contacting the same plurality of antigen-recognition molecules with one or more off-target peptides associated with said target peptide, wherein each of said off-target peptides is presented in a complex with the same MHC molecule as in (a) (MHC-off-target peptide complex); c) selecting one or more antigen-recognition molecules based at least in part on the number of MHC-off-target peptide complexes detectably bound by each of the antigen-recognition molecules; and d) optionally, repeating steps (a)-(c) using selected antigen-recognition molecules.

In some embodiments, the method includes selecting the antigen-recognition molecule(s) that binds to the fewest MHC-off-target peptide complexes. In some embodiments, the selected antigen-recognition molecule(s) detectably bind no more than five (e.g., no more than four, no more than three, no more than two, or no more than one) MHC-off-target peptide complexes, wherein the off-target peptides are expressed in essential, normal tissues.

In some embodiments, the selected antigen-recognition molecule(s) do not detectably bind to any of the MHC-off-target peptide complexes, wherein the off-target peptides are expressed in essential, normal tissues.

In some embodiments, if several selected antigen-recognition molecules bind to at least one MHC-off-target peptide complex, the method may also include a step of comparing the level of binding of the antigen-recognition molecule(s) to the MHC-target peptide complex versus to the MHC-off-target peptide complex(es). The antigen-recognition molecule(s) may likely bind to the MHC-target peptide complex stronger than to the MHC-off-target peptide complex(es). For example, the antigen-recognition molecule(s) may bind to the MHC-target peptide complex about 1000 times, about 500 times, about 200 times, about 100 times, about 90 times, about 80 times, about 70 times, about 60 times, about 50 times, about 40 times, about 30 times, about 20 times, or about 10 times stronger than to the MHC-off-target peptide complex(es). The method may further include selecting the antigen-recognition molecule(s) based at least in part on the MHC-target peptide complex/MHC-off-target peptide complex binding ratio for one or more off-target peptides, wherein a higher MHC-target peptide complex/MHC-off-target peptide complex binding ratio is more desirable.

In some embodiments, selection of antigen-recognition molecules may take into consideration the level of binding of the plurality of antigen-recognition molecules to the MHC-target peptide complex, wherein stronger binding to the MHC-target peptide complex is more desirable. Accordingly, the method may include selecting the antigen-recognition molecule(s) based at least in part on the level of binding to the MHC-target peptide complex.

In some embodiments, the selected antigen-recognition molecules(s) bind to at least one potential secondary target peptide in addition to the MHC-target peptide complex.

Methods to determine binding to pMHC complexes include, for example, surface plasmon resonance (e.g., BIACORE™), or any other biosensor technique, enzyme-linked immunosorbent assay (ELISA), ELISpot, luminescence assay, flow cytometry, chromatography, or microscopy. Alternatively, or in addition, binding may be determined by functional assays in which a biological response is detected upon binding, for example, cytokine release or cell apoptosis.

For example, antibodies and TCRs may be obtained from display libraries in which a pMHC complex of the disclosure is used to pan the library. TCRs can be displayed on the surface of phage particles and yeast particles, for example, and such libraries have been used for the isolation of high affinity variants of TCR derived from T cell clones. TCR phage libraries can be used to isolate TCRs with novel antigen specificity. Such libraries can be constructed with α- and β-chain sequences corresponding to those found in a natural repertoire. However, the random combination of these α- and β-chain sequences, which occurs during library creation, can produce a repertoire of TCRs that may not be naturally occurring.

In some embodiments, a pMHC complex of the disclosure may be used to screen a library of diverse TCRs displayed on the surface of phage particles. The TCRs displayed by said library may not correspond to those contained in a natural repertoire, for example, they may contain α-and β-chain pairing that would not be present in vivo, and/or the TCRs may contain non-natural mutations and/or the TCRs may be in soluble form. Screening may involve panning the phage library with pMHC complexes of the disclosure and subsequently isolating bound phage particles. For this purpose, pMHC complexes may be attached to a solid support, such as a magnet bead, or column matrix and phage bound pMHC complexes isolated, with a magnet, or by chromatography, respectively. The panning steps may be repeated several times. Isolated phage may be further expanded in E. coli cells. Isolated phage particles may be tested for specific binding to pMHC complexes of the disclosure. Binding can be detected using techniques described herein such as, but not limited to, ELISA, or SPR for example using a BIACORE™ instrument. The DNA sequence of the T cell receptor displayed by pMHC binding phage can be further identified by PCR methods.

Alternatively, antigen binding T cells and TCRs can be isolated from fresh blood obtained from patients or healthy donors. Such a method involves stimulating T cells using autologous dendritic cells (DCs), followed by autologous B cells, and then pulsed with a target/off-target peptide disclosed herein. Several rounds of stimulation may be carried out, for example three or four rounds. Activated T cells may then be tested for recognition of the target/off-target peptide by measuring cytokine release in the presence of T2 cells pulsed with the target/off-target peptide of the disclosure (for example using an IFNγ ELISpot assay). Activated cells may then be sorted by fluorescence-activated cell sorting (FACS) using labelled antibodies to detect intracellular cytokine production (e.g. IFNγ), or expression of a cell surface marker (such as CD137). Sorted cells may be expanded and further validated, for example, by ELISpot assay and/or cytotoxicity against target cells and/or staining by peptide-MHC tetramer. The TCR chains from validated clones may then be amplified by rapid amplification of cDNA ends (RACE) and sequenced. An exemplary method for isolation and validation of TCRs specific to a target antigen is described in Moore et al., Sci. Immunol. 6, eabj4026 (2021), which is herein incorporated by reference in its entirety.

TCR screening can also be performed using TCR activation assays. For example, JRT3-T3.5 cells (ATCC TIB-153), a Jurkat subline lacking endogenous TCR surface expression may be utilized as described in Moore et al., Sci. Immunol. 6, eabj4026 (2021), which is herein incorporated by reference in its entirety. T cell receptor alpha (TCRA) and T cell receptor beta (TCRB) sequences of interest can be introduced into the cells by lentiviral transduction, and surface TCR+ cells can be sorted. Antigen-presenting cells (e.g., 293T cells) that are pulsed with a target peptide or off-target peptides can be incubated the TCR-transduced or parental JRT3 cells. A readout (e.g., luciferase activity) is then measured as an indication of TCR-mediated activation.

In some embodiments, monoclonal antibody screening can be performed with cells isolated from the spleens and lymphoid tissue harvested from mice with optimal titers using hybridoma and B cell sorting (BST) platforms. Counter-screening approaches using one or more off-target peptides can help to identify and eliminate B-cells and hybridomas that show cross-reactivity with peptides that form pHLA complexes resembling the targeted complex. For example, antigen positive (Ag⁺) clones that have cross-reactivity to off-target peptides can be identified by examining cell supernatants for antibody binding to cells (e.g., T2 cells) pulsed with the target peptide or off-target peptides using a cell binding assay. As another example, Ag⁺ B-cells can be captured using a biotin-labeled HLA-target peptide complex in the presence of high concentrations of one or more unlabeled, HLA-off-target peptide complexes to enrich for antibodies specific for the HLA-target peptide complex. The antibody variable domains of Ag⁺ B-cells can be subsequently cloned as full-length mAbs and expressed (e.g., in CHO cells) for further screening.

Antibody binding to target/off-target peptides can be determined using ELISA. For example, MHC-target peptide complexes or MHC-off-target peptide complexes can be coated onto a plate (e.g., 96-well microtiter plate). A sample comprising test antibodies can be added to the plate and the reaction can be incubated under a condition to allow binding to occur. The plate is then washed, and a secondary antibody can be then added to the plate to detect the antibodies bound to a MHC-peptide complex. Typically, the secondary antibody can produce a signal that is indicative of the amount of the antibodies bound to the MHC-peptide complex.

In various embodiments of the methods described herein, pMHC complexes may be provided in soluble form, or may be immobilized by attachment to a suitable solid support. Examples of solid supports include, but are not limited to, a bead, a membrane, sepharose, a magnetic bead, a plate, a tube, a column. pMHC complexes may be attached to an ELISA plate, a magnetic bead, or a surface plasmon resonance biosensor chip. Methods of attaching pMHC complexes to a solid support are known to the skilled person, and include, for example, using an affinity binding pair, e.g. biotin and streptavidin, or antibodies and antigens. In some embodiments, pMHC complexes are labeled with biotin and attached to streptavidin-coated surfaces.

In various embodiments of the methods described herein, pMHC complexes may be present on a cell. Such a cell may be a mammalian cell, preferably a cell of the immune system, and a specialized antigen-presenting cell (APC) such as a dendritic cell or a B cell. Other preferred cells include T2 cells. Cells presenting the peptide or pMHC complex of the disclosure may be isolated, preferably in the form of a homogenous population, or provided in a substantially pure form. Such cells may be obtained by pulsing said cells with one or more peptides (e.g., 2 to 10, 2 to 20, 2 to 30, 5 to 25, 5 to 20, or 10 to 15 peptides) of the disclosure, or genetically modifying the cells (via DNA or RNA transfer) to express one or more peptides (e.g., 2 to 10, 2 to 20, 2 to 30, 5 to 25, 5 to 20, or 10 to 15 peptides) of the disclosure. Pulsing involves incubating the cells with the peptide for several hours using peptide concentrations typically ranging from 10⁻⁵ to 10⁻¹² M. Such cells may additionally be transduced with HLA molecules, such as HLA-A*02 to further induce presentation of the peptide(s). Cells may be produced recombinantly.

In various embodiments of the methods described herein, the method is performed in a high-throughput format (e.g., a 96-well plate).

In yet another aspect, provided herein is a method of enriching a sample for antigen-recognition molecules that specifically bind a target peptide, comprising (a) contacting a sample comprising a plurality of antigen-recognition molecules with the target peptide in the presence of one or more off-target peptides associated with said target peptide, wherein each of said target peptide and said one or more off-target peptides is presented in a complex with a major histocompatibility complex (MHC) molecule (MHC-target peptide complex or MHC-off-target peptide complex); and (b) enriching the sample by isolating the antigen-recognition molecules that are bound to the MHC-target peptide complex. The method may further comprise repeating steps (a)-(b) to further enrich the sample.

There are various ways that can allow isolation of the antigen-recognition molecules that are bound to the MHC-target peptide complex. For example, the MHC-target peptide complex may be present on antigen-presenting cells while the MHC-off-target peptide complexes are not present on antigen-presenting cells (e.g., in a soluble form). Alternatively, the MHC-target peptide complex may be immobilized on a solid support while the MHC-off-target peptide complexes may be soluble or immobilized to a different solid support. The MHC-target peptide complex and the MHC-off-target peptide complexes may also be differentially labeled such that specific detection and isolation of the MHC-target peptide complex and the antigen-recognition molecules bound thereon can be achieved. The antigen-recognition molecules may be eluted from the MHC-target peptide complex after isolation.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology are described above with reference to customer devices that may include mobile computing devices. Those skilled in the art will recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXAMPLES

The following examples are provided to further describe some of the embodiments disclosed herein. The examples are intended to illustrate, not to limit, the disclosed embodiments.

### Example 1. Development of Peptide in Groove Similarity Prediction (PIGSPRED) to Predict Off-Targets Associated with Target Peptide-HLA (pHLA) Complexes

Peptide-HLA (pHLA) complexes specifically expressed on cancer cells present a unique set of targets to destroy cancer cells using antibody-based or cell-based therapeutics approaches. However, it is important to take into account the potential off-targets associated with these pHLA complexes to avoid off-target toxicity. While expression of these targets should be specific to cancer cells, it has become increasingly clear that there exists another set of off-targets: pHLA complexes that are highly similar to the target pHLA [Cameron B J et al., Sci Transl Med. 2013; Linette G P et al., Blood. 2013]. The three-dimensional nature of the interactions between pHLA and its cognate therapeutic molecules has made it challenging to ascertain the similarity of pHLA complexes. However, most of the off-target toxicity that has been reported has resulted from off-targets that share the same HLA as the target pHLA, and also contain a peptide that is similar/homologous to the peptide sequence in the target pHLA. The present Example describes the development of a method called PIGSPRED (Peptide in Groove Similarity Prediction) useful in the prediction of such off-targets. After a target pHLA of interest was identified, PIGSPRED predicted off-targets associated with the target, which helped in evaluation of the risk associated with the target at the target selection step, as well as in screening highly specific T-cell Receptor (TCR) or antibodies (Abs). Following input of a target pHLA, the PIGSPRED method operated in the following multi-step fashion:

### 1. Identification of Peptide Positions Important for TOR (T-Cell Receptor) or Antibody Binding

When evaluating similarity/homology of a peptide to the target peptide, it is necessary to evaluate similarity at peptide positions that may be involved in binding interactions with the TCR/antibody. These important positions can be ascertained by analyzing the experimental structures of pHLA in complex with the TCR/antibody typically derived using crystallography or cryo-electron microscopy (cryoEM) techniques. Not only are such structures difficult to obtain, but at the initial target selection stage, where the potential risks associated with the target are evaluated, a TCR/antibody against the target is unavailable. To address this challenge, a computational mutagenesis algorithm using a commercial tool called NetMHCpan, which can predict the binding affinity of the peptide to an HLA using a machine learning model (Jurtz V et al., J Immunol. 2017), was implemented. First, the binding affinity of the peptide to the HLA was predicted. Then the amino acid at each position in the peptide was iteratively mutated to a glycine amino acid, and the affinity of the mutated peptide subsequently predicted. Positions that did not result in the loss of binding affinity to the HLA were flagged for additional consideration, as such positions were not likely to be involved in HLA binding, and therefore were free to interact with the TCR/antibody. Out of these free positions, the ones containing a non-glycine amino acid were identified as the positions important for TCR/antibody binding. As described above, if the structure of a pHLA in complex with the TCR/antibody was available, then the binding motifs in the peptide sequence involved in TCR/antibody interactions could be obtained.

### 2. Identification of Similar Peptides

Canonical human protein sequences were obtained from UniprotKB and similar peptides were identified that were the same length as the target peptide and identical to the target peptide at three or more important amino acid positions. Similar peptides were additionally identified based on experimentally-derived binding motifs in the target peptide sequence. For these similar peptides to be potential off-targets, they would have to be capable of binding the HLA in the target pHLA. Therefore, NetMHCpan was used to predict the binding affinity of each similar peptide to the HLA, and the peptides that were not predicted to bind were discarded from the dataset. A similar peptide may be identical to the target peptide, although from a different protein source as the target peptide.

### 3. Expression Analysis

Once similar peptides were identified, it was necessary to establish that the peptides could potentially cause off-target effects to avoid overestimation of the risk associated with a given target. Therefore, the expression of the peptides in essential, normal tissues or essential cell types was ascertained. For this purpose, the Gene Tissue Expression database or GTEx (gtexportal.org/home/) was queried. The GTEx database version 6, based on genome build GRCh38, contains gene expression data across 51 tissue types from 549 healthy donors. Gene expression values were queried only in those tissue types considered essential, in other words, tissues that cannot be sacrificed for the purpose of saving a patient's life, unlike non-essential tissues such as, e.g., breast, ovary, testes, etc. For each gene, the 95-percentile expression value was calculated in each essential tissue type, followed by calculation of the maximum of that value across all essential tissue types. If the maximum expression value of a gene was greater than 0.5 transcripts per million (TPM), then it was assumed that any similar peptide that was derived from the gene would be a potential off-target.

### 4. Final Output

Once all potential off-targets were identified, a Degree of Similarity (DoS) score was calculated for each off-target peptide to quantify the homology between the off-target and the target peptide. DoS represents the number of identical amino acids at identical positions, or hamming distance, between peptides. Once the DoS scores were determined, the number of off-targets at different threshold values was computed to compare the likelihood of off-target toxicity associated with different targets. Using immunopeptidomics data generated in the present Example, off-target peptides observed in mass-spectrometry experiments were annotated, thereby providing additional evidence for the potential off-targets.

Identification of Potential Off-Targets Associated with an HLA-A2 Peptide
Identification of Potential Off-Targets Associated with a First MAGEA4 Target Peptide: GVYDGREHTV (SEQ ID NO: 1)

Tables 1A-3C illustrate results of applying the PIGSPRED method inputting MAGEA4₂₃₀₋₂₃₉ target GVYDGREHTV (SEQ ID NO: 1)-HLA-A*02:01 at step 102.

Table 1A includes a number of potential high risk off-target peptides associated with the MAGEA4₂₃₀₋₂₃₉ target GVYDGREHTV (SEQ ID NO: 1)-HLA-A*02:01 at different DoS threshold values. The number of potential high risk off-target peptides identified computationally are provided. Important positions were predicted computationally at step 104 to be positions 4 and 6-9. The table also provides the number of computationally predicted potential high risk off-target peptides that were also identified in immunopeptidomics mass-spectrometry data.

TABLE 1A

| | DoS >= | DoS >= | DoS >= | DoS >= | DoS >= | DoS >= | DoS >= | DoS >= |
|---|---|---|---|---|---|---|---|---|
| Identified | 3 | 4 | 5 | 6 | 7 | 8 | 9 | 10 |
| Computationally | 184 | 89 | 26 | 6 | 1 | 0 | 0 | 0 |
| By Mass Spec | 12 | 5 | 3 | 1 | 1 | 0 | 0 | 0 |

Table 1B includes potential off-targets associated with the MAGEA4₂₃₀₋₂₃₉ target GVYDGREHTV (SEQ ID NO: 1)-HLA-A*02:01 at a DoS greater than or equal to 6. The predicted binding affinity of each off-target is represented by the half maximal inhibitory concentration (IC₅₀) value and a binding affinity percentile rank value (% Rank_BA in NetMHCpan). The mRNA level (TMP from GTEx) in the normal tissue with the highest expression for each off-target gene is provided, as well as the top three highly expressing normal tissues. Peptides found in mice may be useful for conducting antigen-recognition molecule binding studies (e.g., screenings) in mice. Potential off-target (1) GLADGRTHTV (SEQ ID NO: 2) was detected in mass-spectrometry experiments, while other potential off-targets in the table were not. Potential off-target peptides GLYDGREHSV (SEQ ID NO: 73, DoS=8) from the MAGEA8 gene (TPM=0.4, not expressed in mouse) and GLYDGMEHLI (SEQ ID NO: 74, DOS=6) from the MAGEA10 gene (TPM=0.1, not expressed in mouse) were filtered from the list of potential off-target peptides in Table 1B due to their low expression levels in essential normal tissue. However, as both MAGEA8 and MAGEA10 are cancer/testis (CT) antigens that may be useful for targeting in cancer immunotherapy applications, another working list comprising such potential secondary targets may be prepared, and additional testing (e.g., antigen-recognition molecule screening) may optionally be performed on one or more such potential secondary targets to assess whether cross-reactivity with those peptides could provide a beneficial therapeutic effect.

TABLE 1B

| | | | | percentile | Off | | Normal | | |
|---|---|---|---|---|---|---|---|---|---|
| | | | | rank | target | | tissue | In | Mass |
| No | Off-target | DoS | IC₅₀ | value | gene | TPM | samples | mouse | Spec |
| 1 | GLADGRTHTV | 7 | 46.8 | 0.6 | THBS3 | 67.6 | Nerve, | No | Yes |
| | (SEQ ID NO: 2) | | | | | | Colon, | | |
| | | | | | | | Esophagus | | |
| 2 | GVPDCRIFTV | 6 | 271.3 | 2 | LPIN2 | 78.6 | Liver, | Yes | No |
| | (SEQ ID NO: 3) | | | | | | Small | | |
| | | | | | | | Intestine, | | |
| | | | | | | | Kidney | | |
| 3 | SVYDAREFSV | 6 | 77.6 | 0.9 | SLC25A19 | 59.3 | Adrenal | No | No |
| | (SEQ ID NO: 4) | | | | | | Gland, | | |
| | | | | | | | Spleen, | | |
| | | | | | | | Lung | | |
| 4 | GLSDGQWHTV | 6 | 25.3 | 0.3 | CELSR3 | 45.4 | Brain, | Yes | No |
| | (SEQ ID NO: 5) | | | | | | Pituitary, | | |
| | | | | | | | Adrenal | | |
| | | | | | | | Gland | | |
| 5 | GVFDNCSHTV | 6 | 107.5 | 1.1 | PAPPA2 | 32.8 | Kidney, | No | No |
| | (SEQ ID NO: 6) | | | | | | Pituitary, | | |
| | | | | | | | Stomach | | |
| 6 | KVSDGHFHTV | 6 | 236.5 | 1.8 | FAT4 | 16.4 | Blood | No | No |
| | (SEQ ID NO: 7) | | | | | | Vessel, | | |
| | | | | | | | Colon, | | |
| | | | | | | | Lung | | |

Table 2A includes the number of potential off-targets associated with the MAGEA4₂₃₀₋₂₃₉ target GVYDGREHTV (SEQ ID NO: 1)-HLA-A*02:01 at different DoS threshold values. An experimentally derived binding motif, XXXDXREXXX (SEQ ID NO: 8), was used where X represents any amino acid.

TABLE 2A

| | DoS >= | DoS >= | DoS >= | DoS >= | DoS >= | DoS >= | DoS >= | DoS >= |
|---|---|---|---|---|---|---|---|---|
| Identified | 3 | 4 | 5 | 6 | 7 | 8 | 9 | 10 |
| Computationally | 11 | 6 | 1 | 1 | 0 | 0 | 0 | 0 |
| By Mass Spec | 1 | 0 | 0 | 0 | 0 | 0 | 0 | 0 |

Table 2B includes potential off-targets associated with the MAGEA4₂₃₀₋₂₃₉ target GVYDGREHTV (SEQ ID NO: 1)-HLA-A*02:01 at DoS>=3. An experimentally derived binding motif, XXXDXREXXX (SEQ ID NO: 8), was used where X represents any amino acid. Potential off-target (9) ALVDQRELYL (SEQ ID NO: 9) was detected in mass spectrometry experiments, while other potential off-targets in the table were not.

TABLE 2B

| | | | | percentile | Off | | Normal | | |
|---|---|---|---|---|---|---|---|---|---|
| | | | | rank | target | | tissue | In | Mass |
| No | Off-target | DoS | IC₅₀ | value | gene | TPM | samples | mouse | Spec |
| 1 | SVYDAREFSV | 6 | 77.6 | 0.9 | SLC25 | 59.3 | Adrenal | No | No |
| | (SEQ ID NO: | | | | A19 | | Gland, | | |
| | 4) | | | | | | Spleen, | | |
| | | | | | | | Lung | | |
| 2 | ALLDYREDG | 4 | 59.6 | 0.7 | TPT1 | 11657.3 | Muscle, | Yes | No |
| | V (SEQ ID NO: | | | | | | Adipose | | |
| | 11) | | | | | | Tissue, | | |
| | | | | | | | Salivary | | |
| | | | | | | | Gland | | |
| 3 | CLIDDREMPV | 4 | 27.1 | 0.4 | IPO7 | 136.5 | Muscle, | Yes | No |
| | (SEQ ID NO: | | | | | | Bladder, | | |
| | 12) | | | | | | Blood | | |
| | | | | | | | Vessel | | |
| 4 | LLDDNRELL | 4 | 77.4 | 0.9 | MDN1 | 20.1 | Brain, | No | No |
| | V (SEQ ID NO: | | | | | | Nerve, | | |
| | 13) | | | | | | Skin | | |
| 5 | VLVDDRECP | 4 | 130.7 | 1.3 | PFAS | 17 | Spleen, | No | No |
| | V (SEQ ID NO: | | | | | | Nerve, | | |
| | 14) | | | | | | Lung | | |
| 6 | ILIDIREFTL | 4 | 14.3 | 0.2 | ZNF781 | 13.7 | Brain, | No | No |
| | (SEQ ID NO: | | | | | | Pituitary, | | |
| | 15) | | | | | | Colon | | |
| 7 | VLIDIREYWM | 3 | 101.7 | 1.1 | SUB1 | 378.4 | Spleen, | No | No |
| | (SEQ ID NO: | | | | | | Adipose | | |
| | 16) | | | | | | Tissue, | | |
| | | | | | | | Lung | | |
| 8 | YSMDIREFQL | 3 | 101.1 | 1.1 | BPI | 198.7 | Blood, | No | No |
| | (SEQ ID NO: | | | | | | Spleen, | | |
| | 17) | | | | | | Lung | | |
| 9 | ALVDQRELY | 3 | 88 | 1 | TRAPP | 179.2 | Adrenal | Yes | Yes |
| | L (SEQ ID NO: | | | | C2L | | Gland, | | |
| | 9) | | | | | | Lung, | | |
| | | | | | | | Adipose | | |
| | | | | | | | Tissue | | |
| 10 | YLADEREDL | 3 | 94.6 | 1 | SLC4A2 | 111.9 | Stomach, | No | No |
| | L (SEQ ID NO: | | | | | | Colon, | | |
| | 18) | | | | | | Lung | | |
| 11 | ILLDNREYLA | 3 | 121.9 | 1.2 | TTC26 | 7.2 | Lung, | Yes | No |
| | (SEQ ID NO: | | | | | | Pituitary, | | |
| | 19) | | | | | | Nerve | | |

Table 3A includes the number of potential off-targets associated with the MAGEA4₂₃₀₋₂₃₉ target GVYDGREHTV (SEQ ID NO: 1)-HLA-A*02:01 at different DoS threshold values. An experimentally derived binding motif, XX[YFWM][DENQ][GA][R][DENQ]XXX (SEQ ID NO: 10), was used where X represents any amino acid.

TABLE 3A

| | DoS >= | DoS >= | DoS >= | DoS >= | DoS >= | DoS >= | DoS >= | DoS >= |
|---|---|---|---|---|---|---|---|---|
| Identified | 3 | 4 | 5 | 6 | 7 | 8 | 9 | 10 |
| Computationally | 5 | 1 | 1 | 1 | 0 | 0 | 0 | 0 |
| By Mass Spec | 1 | 0 | 0 | 0 | 0 | 0 | 0 | 0 |

Table 3B includes potential off-targets associated with the MAGEA4₂₃₀₋₂₃₉ target GVYDGREHTV (SEQ ID NO: 1)-HLA-A*02:01 at DoS>=2. An experimentally derived binding motif, XX[YFWM][DENQ][GA][R][DENQ]XXX (SEQ ID NO: 10), was used where X represents any amino acid. Potential off-target (5) ALVDQRELYL (SEQ ID NO: 9) was detected in mass spectrometry experiments, while other potential off-targets in the table were not.

TABLE 3B

| | | | | percentile | Off | | Normal | | |
|---|---|---|---|---|---|---|---|---|---|
| | | | | rank | target | | tissue | In | Mass |
| No | Off-target | DoS | IC₅₀ | value | gene | TPM | samples | mouse | Spec |
| 1 | SVYDAREFSV | 6 | 77.6 | 0.9 | SLC25A19 | 59.3 | Adrenal | No | No |
| | (SEQ ID NO: 4) | | | | | | Gland, | | |
| | | | | | | | Spleen, | | |
| | | | | | | | Lung | | |
| 2 | MLWQGRDD | 3 | 50.3 | 0.6 | ABRA | 165.2 | Muscle, | Yes | No |
| | HV (SEQ ID | | | | | | Heart, | | |
| | NO: 20) | | | | | | Salivary | | |
| | | | | | | | Gland | | |
| 3 | IIFDARQNSV | 3 | 261. | 2 | MTMR1 | 30.9 | Skin, | No | No |
| | (SEQ ID NO: | | 3 | | | | Brain, | | |
| | 21) | | | | | | Spleen | | |
| 4 | LLFQGRNPGV | 3 | 34 | 0.5 | ADAMTS1 | 15.1 | Brain, | Yes | No |
| | (SEQ ID NO: | | | | 6 | | Adipose | | |
| | 22) | | | | | | Tissue, | | |
| | | | | | | | Kidney | | |
| 5 | FMFEGREIKL | 3 | 8 | 0.1 | DNAH6 | 5.7 | Lung, | No | Yes |
| | (SEQ ID NO: | | | | | | Pituitary, | | |
| | 23) | | | | | | Brain | | |
| 6 | QMFDARNM | 2 | 164. | 1.5 | TUBB6 | 439.1 | Esophagus, | No | No |
| | MA (SEQ ID | | 4 | | | | Adipose | | |
| | NO: 24) | | | | | | Tissue, | | |
| | | | | | | | Colon | | |
| 7 | YMYQARDLA | 2 | 44.8 | 0.6 | DYSF | 342.9 | Blood, | No | No |
| | A (SEQ ID NO: | | | | | | Spleen, | | |
| | 25) | | | | | | Muscle | | |
| 8 | YMFEAREFLR | 2 | 69.3 | 0.8 | SND1 | 155.6 | Pancreas, | Yes | No |
| | (SEQ ID NO: | | | | | | Lung, | | |
| | 26) | | | | | | Bladder | | |
| 9 | LLMDARDLV | 2 | 27.7 | 0.4 | METTL17 | 91.2 | Adrenal | No | No |
| | L (SEQ ID NO: | | | | | | Gland, | | |
| | 27) | | | | | | Spleen, | | |
| | | | | | | | Small | | |
| | | | | | | | Intestine | | |
| 10 | KMMEGRNSS | 2 | 73.7 | 0.9 | BRD1 | 64.4 | Brain, | Yes | No |
| | I (SEQ ID NO: | | | | | | Skin, | | |
| | 28) | | | | | | Nerve | | |

Identification of Potential Off-Targets Associated with a Second MAGEA4 Target Peptide: KVLEHVVRV (SEQ ID NO: 48)

Tables 4A-4B illustrates results of applying the PIGSPRED method when inputting an alternative MAGEA4 peptide-derived pMHC target, MAGEA4₂₈₆₋₂₉₄ target KVLEHVVRV (SEQ ID NO: 48)-HLA-A*02:01, at step 102.

Table 4A includes a number of potential high risk off-target peptides associated with the MAGEA4₂₈₆₋₂₉₄ target KVLEHVVRV (SEQ ID NO: 48)-HLA-A*02:01 at different DoS threshold values. The number of potential high risk off-target peptides identified computationally are provided. Important positions were predicted computationally at step 104. The table also provides the number of computationally predicted potential high risk off-target peptides that were also identified in immunopeptidomics mass-spectrometry data.

TABLE 4A

| | DoS >= | DoS >= | DoS >= | DoS >= | DoS >= | DoS >= | DoS >= |
|---|---|---|---|---|---|---|---|
| Identified | 3 | 4 | 5 | 6 | 7 | 8 | 9 |
| Computationally | 3794 | 1531 | 251 | 23 | 0 | 0 | 0 |
| By Mass Spec | 882 | 387 | 75 | 11 | 0 | 0 | 0 |

Table 4B includes potential off-targets associated with the target at a DoS greater than or equal to 6. Eleven different potential off-targets were detected in mass-spectrometry experiments, while other potential off-targets in the table were not. Off-target peptide KVLEHVVRV (SEQ ID NO: 49, DoS=9) from the MAGEA8 gene (TPM=0.4, not expressed in mouse), which is identical to the MAGEA4₂₈₆₋₂₉₄ target peptide, was filtered from the list of potential off-target peptides in Table 4B due to its low expression levels in essential normal tissue.

TABLE 4B

| | | | | percentile | Off | | Normal | | |
|---|---|---|---|---|---|---|---|---|---|
| | | | | rank | target | | tissue | In | Mass |
| No | Off-target | DoS | IC₅₀ | value | gene | TPM | samples | mouse | Spec |
| 1 | KVMCHVR | 6 | 129.9 | 1.28 | AOX1 | 649.2 | Liver, | Yes | No |
| | RV (SEQ | | | | | | Adrenal | | |
| | ID NO: 50) | | | | | | Gland, | | |
| | | | | | | | Adipose | | |
| | | | | | | | Tissue | | |
| 2 | KVLERVN | 6 | 21.4 | 0.31 | PSME2 | 394.4 | Spleen, | Yes | Yes |
| | AV (SEQ | | | | | | Small | | |
| | ID NO: 51) | | | | | | Intestine, | | |
| | | | | | | | Adrenal | | |
| | | | | | | | Gland | | |
| 3 | NVTEYVV | 6 | 172.8 | 1.53 | GTF2F1 | 132.7 | Brain, | Yes | Yes |
| | RV (SEQ | | | | | | Pituitary, | | |
| | ID NO: 52) | | | | | | Bladder | | |
| 4 | FLLETVVR | 6 | 2.8 | 0.01 | RABGA | 113.6 | Heart, | Yes | Yes |
| | V (SEQ ID | | | | PIL | | Esophagus, | | |
| | NO: 53) | | | | | | Brain | | |
| 5 | KVLEQPV | 6 | 111.6 | 1.16 | MRPL3 | 109.2 | Adrenal | No | Yes |
| | VV (SEQ | | | | 7 | | Gland, | | |
| | ID NO: 54) | | | | | | Muscle, | | |
| | | | | | | | Liver | | |
| 6 | MVLEHPA | 6 | 30.8 | 0.45 | CARD8 | 103.4 | Spleen, | No | No |
| | RV (SEQ | | | | | | Blood, | | |
| | ID NO: 55) | | | | | | Small | | |
| | | | | | | | Intestine | | |
| 7 | KVLQNVL | 6 | 117.8 | 1.20 | PLEKH | 102.5 | Lung, | No | No |
| | RV (SEQ | | | | H2 | | Nerve, | | |
| | ID NO: 56) | | | | | | Adipose | | |
| | | | | | | | Tissue | | |
| 8 | KVLGIVV | 6 | 7.4 | 0.08 | CNOT1 | 91.6 | Esophagus, | Yes | Yes |
| | GV (SEQ | | | | | | Spleen, | | |
| | ID NO: 57) | | | | | | Small | | |
| | | | | | | | Intestine | | |
| 9 | KQMEHVQ | 6 | 5.2 | 0.04 | THOC2 | 52.8 | Esophagus, | Yes | No |
| | RV (SEQ | | | | | | Lung, | | |
| | ID NO: 58) | | | | | | Bladder | | |
| 10 | KVLESGV | 6 | 64.1 | 0.79 | TENM2 | 46.2 | Heart, | Yes | No |
| | NV (SEQ | | | | | | Nerve, | | |
| | ID NO: 59) | | | | | | Brain | | |
| 11 | KVLEILHR | 6 | 5.9 | 0.06 | HERC4 | 38.7 | Spleen, | Yes | Yes |
| | V (SEQ ID | | | | | | Nerve, | | |
| | NO: 60) | | | | | | Lung | | |
| 12 | GVLEHIGR | 6 | 164.4 | 1.48 | SLITRK | 26.1 | Adrenal | Yes | No |
| | V (SEQ ID | | | | 4 | | Gland, | | |
| | NO: 61) | | | | | | Muscle, | | |
| | | | | | | | Brain | | |
| 13 | AVEEHVV | 6 | 201.4 | 1.68 | TEP1 | 25.7 | Small | No | Yes |
| | SV (SEQ ID | | | | | | Intestine, | | |
| | NO: 62) | | | | | | Spleen, | | |
| | | | | | | | Colon | | |
| 14 | KVIECNV | 6 | 34.1 | 0.49 | CAD | 24.6 | Nerve, | Yes | No |
| | RV (SEQ | | | | | | Pituitary, | | |
| | ID NO: 63) | | | | | | Spleen | | |
| 15 | VVLEPVE | 6 | 225.6 | 1.79 | PASK | 23.4 | Brain, | Yes | No |
| | RV (SEQ | | | | | | Small | | |
| | ID NO: 64) | | | | | | Intestine, | | |
| | | | | | | | Pituitary | | |
| 16 | KVLECCH | 6 | 36.6 | 0.52 | RNF182 | 21.8 | Brain, | Yes | No |
| | RV (SEQ | | | | | | Lung, | | |
| | ID NO: 65) | | | | | | Pituitary | | |
| 17 | KILEDVV | 6 | 5.4 | 0.04 | TPX2 | 20.5 | Esophagus, | Yes | Yes |
| | GV (SEQ | | | | | | Colon, | | |
| | ID NO: 66) | | | | | | Spleen | | |
| 18 | KVLEGVV | 6 | 60.4 | 0.76 | NBAS | 18.9 | Pituitary, | No | No |
| | AA (SEQ | | | | | | Nerve, | | |
| | ID NO: 67) | | | | | | Brain | | |
| 19 | KVLETLV | 6 | 6.2 | 0.06 | HEATR | 15.4 | Adrenal | No | Yes |
| | TV (SEQ ID | | | | 5A | | Gland, | | |
| | NO: 68) | | | | | | Nerve, | | |
| | | | | | | | Pituitary | | |
| 20 | KVLESVF | 6 | 12.4 | 0.17 | PCGF6 | 13.8 | Colon, | Yes | Yes |
| | RI (SEQ ID | | | | | | Bladder, | | |
| | NO: 69) | | | | | | Nerve | | |
| 21 | KVLEHVP | 6 | 13.5 | 0.18 | SMIM1 | 9.9 | Bladder, | Yes | Yes |
| | LL (SEQ ID | | | | 1A | | Stomach, | | |
| | NO: 70) | | | | | | Nerve | | |
| 22 | KVLEEVD | 6 | 14.5 | 0.20 | CCDC1 | 6.7 | Kidney, | No | No |
| | FV (SEQ ID | | | | 48 | | Pituitary, | | |
| | NO: 71) | | | | | | Brain | | |
| 23 | EVLENVF | 6 | 205.5 | 1.70 | HESX1 | 2.6 | Kidney, | Yes | No |
| | RV (SEQ | | | | | | Nerve, | | |
| | ID NO: 72) | | | | | | Pituitary | | |

Both MAGE-A4230-239 and MAGE-A4₂₈₆₋₂₉₄ were computationally predicted to bind HLA-A02 with relatively high affinity (IC₅₀=560.08 nM and 8.52 nM, respectively). Application of the PIGSPRED method as illustrated in Tables 1B and 4B identified 6 and 23 peptides with DoS>6 expressed in normal essential tissue for MAGE-A4230-239 and MAGE-A4286-294, respectively, suggesting that targeting the former may have a lower potential for producing off-target toxicity. Notably, both MAGE-A4230-239 and MAGE-A4₂₈₆₋₂₉₄ share a high DoS with peptides derived from MAGE-A8 (DoS of 9 and 8, respectively), another CT antigen that shows negligible expression in essential normal tissue. As described above, with respect to Table 1B, such off-targets may be separately tabulated and, optionally, tested for binding of target antigen-recognition molecules.

Identification of Potential Off-Targets Associated with an HLA-A1 Peptide

The in-silico computational strategy as detailed above was used to identify potential off-targets associated with a target peptide associated with HLA-A*01:01, MAGEA3₁₆₈₋₁₇₆ target EVDPIGHLY (SEQ ID NO: 29)-HLA-A*01:01 at step 102.

Table 5A shows the number of potential off-targets associated with the MAGEA3₁₆₈₋₁₇₆ target EVDPIGHLY (SEQ ID NO: 29)-HLA-A*01:01 at different DoS threshold values. Important positions were predicted computationally at step 104.

TABLE 5A

| | DoS >= | DoS >= | DoS >= | DoS >= | DoS >= | DoS >= | DoS >= |
|---|---|---|---|---|---|---|---|
| Identified | 3 | 4 | 5 | 6 | 7 | 8 | 9 |
| Computationally | 1236 | 988 | 141 | 10 | 2 | 0 | 0 |
| By Mass Spec | 183 | 168 | 34 | 4 | 2 | 0 | 0 |

Table 5B shows a representative list of the potential off-targets associated with the MAGEA3₁₆₈₋₁₇₆ target EVDPIGHLY (SEQ ID NO: 29)-HLA-A*01:01 at DoS>=5. Additional off-targets with DoS>=5 or below are not shown (indicated with “ . . . ”). Potential off-targets (1) (2), (3), (10), (12), (14)-(16) and (18) were detected in mass-spectrometry experiments, while other potential off-targets in the table were not.

TABLE 5B

| | | | | percentile | Off- | | | | |
|---|---|---|---|---|---|---|---|---|---|
| | | | | rank | target | | Normal tissue | In | Mass |
| No | Off-target | DoS | IC₅₀ | value | gene | TPM | samples | mouse | Spec |
| 1 | EVGPIFHLY | 7 | 972 | 0.5 | FGD5 | 44.1 | Lung, Spleen, | No | Yes |
| | (SEQ ID NO: | | | | | | Adipose Tissue | | |
| | 30) | | | | | | | | |
| 2 | EVVRIGHLY | 7 | 3723.3 | 1.2 | MAGEA12 | 0.6 | Brain | No | Yes |
| | (SEQ ID NO: | | | | | | | | |
| | 31) | | | | | | | | |
| 3 | YVDSEGHLY | 6 | 7.8 | 0 | CAVI | 819 | Adipose Tissue, | Yes | Yes |
| | (SEQ ID NO: | | | | | | Lung, Bladder | | |
| | 32) | | | | | | | | |
| 4 | EVDKIGRGY | 6 | 1275.8 | 0.6 | LONP1 | 366.2 | Adrenal Gland, | Yes | No |
| | (SEQ ID NO: | | | | | | Liver, Pituitary | | |
| | 33) | | | | | | | | |
| 5 | EVDQIYHLA | 6 | 3749.7 | 1.2 | UXS1 | 73.9 | Kidney, Nerve, | Yes | No |
| | (SEQ ID NO: | | | | | | Lung | | |
| | 34) | | | | | | | | |
| 6 | VVDNIDHLY | 6 | 11.4 | 0 | COP1 | 48 | Blood, Bladder, | Yes | No |
| | (SEQ ID NO: | | | | | | Skin | | |
| | 35) | | | | | | | | |
| 7 | EVDELVHLY | 6 | 24 | 0 | FSD2 | 47.8 | Muscle, Heart, | No | No |
| | (SEQ ID NO: | | | | | | Salivary Gland | | |
| | 36) | | | | | | | | |
| 8 | EVSPDGELY | 6 | 1203.3 | 0.5 | INPP5B | 47.3 | Blood Vessel, | Yes | No |
| | (SEQ ID NO: | | | | | | Esophagus, | | |
| | 37) | | | | | | Bladder | | |
| 9 | EDDVIEHLY | 6 | 1487.9 | 0.6 | KIAA1109 | 36.2 | Pituitary, | No | No |
| | (SEQ ID NO: | | | | | | Brain, | | |
| | 38) | | | | | | Nerve | | |
| 10 | EVDPTSHSY | 6 | 45.8 | 0 | MAGEA11 | 0.5 | Skin | No | Yes |
| | (SEQ ID NO: | | | | | | | | |
| | 39) | | | | | | | | |
| 11 | WNIPIGLLY | 5 | 2426.2 | 0.9 | TF | 4445.3 | Liver, Brain, | No | No |
| | (SEQ ID NO: | | | | | | Salivary Gland | | |
| | 40) | | | | | | | | |
| 12 | FVGEIGDLY | 5 | 140.5 | 0.1 | APCS | 2389.1 | Liver, Pancreas, | No | Yes |
| | (SEQ ID NO: | | | | | | Kidney | | |
| | 41) | | | | | | | | |
| 13 | ELDPEGSLH | 5 | 3525.8 | 1.2 | ITGA5 | 1521.3 | Blood Vessel, | No | No |
| | (SEQ ID NO: | | | | | | Colon, Bladder | | |
| | 42) | | | | | | | | |
| 14 | DVNGIRHLY | 5 | 2339.4 | 0.9 | MMP9 | 1187.8 | Blood, Spleen, | No | Yes |
| | (SEQ ID NO: | | | | | | Lung | | |
| | 43) | | | | | | | | |
| 15 | EVAPAGASY | 5 | 3473.7 | 1.2 | NOP53 | 1043 | Skin, Colon, | No | Yes |
| | (SEQ ID NO: | | | | | | Adipose Tissue | | |
| | 44) | | | | | | | | |
| 16 | ELDPIQKLF | 5 | 3389 | 1.1 | ATP5PF | 800 | Heart, Muscle, | No | Yes |
| | (SEQ ID NO: | | | | | | Kidney | | |
| | 45) | | | | | | | | |
| 17 | ESLPKDHLY | 5 | 635.8 | 0.4 | TTN | 745 | Muscle, Heart, | No | No |
| | (SEQ ID NO: | | | | | | Salivary Gland | | |
| | 46) | | | | | | | | |
| 18 | ESDPIVAQY | 5 | 20.2 | 0 | TTN | 745 | Muscle, Heart, | No | Yes |
| | (SEQ ID NO: | | | | | | Salivary Gland | | |
| | 47) | | | | | | | | |

Of note, peptide ESDPIVAQY (SEQ ID NO: 47) from the muscle protein Titin was identified as one of the highly ranked potential off-target peptides (see 18th peptide in Table 5B) for the MAGEA3₁₆₈₋₁₇₆ target. Expression of this peptide is also observed in heart tissues. Interestingly, this peptide was reported in Cameron B J et al., Sci Transl Med. 2013 as a cross-reactive target for engineered MAGE A3-directed T cells and the most likely cause of in vivo cardiac toxicity observed in the clinical trials evaluating the engineered MAGE A3-directed T cells. This result demonstrates that the prediction method described herein can accurately identify potential off-targets for a pHLA complex and may be applied to mitigate the risk of off-target toxicity in future clinical investigations.

### Example 2. Binding of Anti-HLA-A2: MAGEA4₂₃₀₋₂₃₉ Antibodies to T2 Cells Pulsed with MAGEA4₂₃₀₋₂₃₉ and Related Off-Target Peptides

Ab A and Ab B are monoclonal antibodies (mAb) with a human fragment crystallizable (Fc) region that recognize amino acids 230-239 of MAGEA4 when in complex with the Human Leukocyte Antigen (HLA) Class I allele HLA-A*02:01.

Cell surface binding of the anti-HLA-A2: MAGEA4₂₃₀₋₂₃₉ antibodies to HLA-A*02:01 positive T2 (174 CEM.T2) cells was assessed in a flow cytometry-based peptide pulsing assay. To pulse, 1×10⁶ T2 cells were incubated with 10 μg/ml of human (h) β2M (EMD Millipore Cat #475828) and 100 μg/ml of a MAGEA4₂₃₀₋₂₃₉ peptide in 1 ml of AIM V medium (Gibco. Cat #31035025) for 16 h at 37° C. Cells were washed in staining buffer (PBS without Calcium and Magnesium (Corning, Ref #21-031-CV)+2% FBS (Seradigm, Lot #238β15)), harvested using cell dissociation buffer (Millipore, Cat #S-004-C), and resuspended in staining buffer. Pulsed cells (200,000) were plated in 96 well V-Bottom plates (Axygen, Cat #P-96-450V-C-S) and stained with three-fold serial dilutions (1.7 pM-100 nM) of Ab A, Ab B, a non-binding isotype control antibody, or an HLA-A2 antibody (data not shown in Table 6) for 30 mins at 4° C. Cells were then washed once in staining buffer, and incubated with an Alexa Fluor 647 conjugated Fab′2 anti-mouse Fc-specific secondary antibody (Jackson ImmunoResearch, Cat #115-606-071) at 5 μg/ml for 30 mins at 4° C. Finally, cells were stained with a green fluorescent viability dye (Molecular Probes Cat #L-34970 reconstituted in 50 μl DMSO) at a concentration of 1:1000. Cells were then washed and fixed using a 50% solution of BD Cytofix (BD, Cat #554655) diluted in PBS. Samples were run on an intellicyt iQue flow cytometer (Intellicyt) and results were analyzed using Forecyte analysis software (Intellicyte) to calculate the mean fluorescent intensity (MFI) after gating for live cells. MFI values were plotted in Graphpad Prism using a four-parameter logistic equation over a 12-point response curve to calculate EC₅₀ values. The secondary antibody alone (i.e. no primary antibody) for each dose-response curve is also included in the analysis as a continuation of the three-fold serial dilution and is represented as the lowest dose. The signal to noise (S/N) was determined by taking the ratio of the highest MFI on the dose response curve to the MFI in the secondary alone wells. The EC₅₀ values (M) and max S/N are shown in Table 6. Ab A bound with an EC₅₀ of 4.7 nM and a max S/N of 365.9, Ab B bound with an EC₅₀ of 1.3 nM and a max S/N of 511.7. The isotype control antibody had minimal binding with a S/N of 13.4.

TABLE 6
Binding of Anti-HLA-A2:MAGEA4₂₃₀₋₂₃₉ antibody to T2
cells pulsed with MAGEA4₂₃₀₋₂₃₉ via flow cytometry

| mAb | EC₅₀ | Max S/N |
|---|---|---|
| Ab A | 4.7e−9 | 365.9 |
| Ab B | 1.3e−9 | 511.7 |
| Isotype Control Ab | ND | 13.4 |

ND = EC₅₀ values could not be determined with accuracy because the binding did not reach saturation within the tested antibody concentration range

An in-silico computational strategy described in Example 1 above identified several MAGEA4-related peptides predicted to form complexes with HLA-A*02:01. The identified peptides are summarized in Table 1B. Binding of the two HLA-A2: MAGEA4₂₃₀₋₂₃₉ antibodies (Ab A and Ab B), a non-binding isotype control antibody and an HLA-A2 antibody to these related peptides was assessed in the T2 pulsing assay described above. S/N is displayed as a ratio of pulsed to unpulsed (no peptide) cells. Peptides were determined to load if HLA-A2 binding had a signal to noise greater than 1. As summarized in Table 7, both Ab A and Ab B bound MAGEA4 peptide with S/N values of 512.9 for Ab A and 747.2 for Ab B. Ab A was highly specific for the MAGEA4 230-239 peptide, while Ab B had strong binding to the off-target peptide LPIN2 with a S/N value of 27.5. No detectable binding to the remaining peptides was observed and the control antibody binding was <4.3 for all tested peptides.

TABLE 7
Binding of anti-HLA-A2:MAGEA4₂₃₀₋₂₃₉ antibodies T2 cells
pulsed with related peptides via flow cytometry

| Peptide | Antibody | | | |
|---|---|---|---|---|
| (Gene name + | | | | Isotype |
| peptide location) | Ab A | Ab B | HLA-A2 Ab | Control Ab |
| No Peptide | 1.0 | 1.0 | 1 | 1 |
| MAGEA4 ₂₃₀₋₂₃₉ | 512.9 | 747.2 | 3.0 | 4.3 |
| THBS3 ₁₁₆₋₁₂₅ | 1.0 | 1.2 | 4.5 | 1.0 |
| LPIN2 ₈₂₃₋₈₃₂ | 0.9 | 27.5 | 6.7 | 0.9 |
| SLC25A19 ₁₁₁₋₁₂₀ | 0.8 | 1.0 | 2.3 | 0.9 |
| CELSR3 ₁₅₉₄₋₁₆₀₃ | 0.8 | 0.9 | 4.9 | 0.9 |
| PAPPA2 ₃₀₇₋₃₁₆ | 0.9 | 1.0 | 4.1 | 0.9 |
| FAT4 ₄₀₅₃₋₄₀₆₂ | 1.0 | 1.5 | 4.6 | 1.1 |

### Example 3. Application of PIGSPRED for Target Discovery and Prioritization

Cancer-specific pHLA complexes can be identified by ascertaining genes that are specifically expressed in cancer tissue. For this purpose, public databases including, for example,

The Cancer Genome Atlas (TCGA) and Genome Tissue Expression Database (GTEx) were used. A gene that was expressed in a cancer type at 75-percentile transcripts per million (TPM) value >2 and was negligibly expressed in all essential, normal tissues or essential cell types in accordance with the GTEx was classified as a cancer-specific gene. The canonical protein sequence corresponding to the cancer-specific gene was derived from the UniProtKB database, and was used to predict the potential 8-12 mer peptide sequences that were predicted to bind the HLA of interest. The predictions were performed using NetMHCpan tool. Once cancer-specific pHLAs were identified, PIGSPRED was used to calculate the number of potential off-targets associated with each cancer-specific pHLA. The number of potential off-targets was representative of the likelihood of off-target toxicity associated with a target, and was therefore used to rank the list of pHLA targets and to prioritize the targets for therapeutic development. After the target selection and the generation of therapeutic molecules that bind the target, the off-targets predicted by PIGSPRED play an important role in experimental screening of therapeutic molecules that do not bind the off-targets. The most specific therapeutic molecules are thus selected for further development.

### REFERENCES

Boehm K M, Bhinder B, Raja V J, Dephoure N, and Elemento O. Predicting peptide presentation by major histocompatibility complex class I: an improved machine learning approach to the immunopeptidome. BMC Bioinformatics. 2019 Jan. 5; 20 (1): 7. doi: 10.1186/s12859-018-2561-z.
Jurtz V, Paul S, Andreatta M, Marcatili P, Peters B, and Nielsen M. NetMHCpan-4.0: Improved Peptide-MHC Class I Interaction Predictions Integrating Eluted Ligand and Peptide Binding Affinity Data. J Immunol. 2017 Nov. 1; 199 (9): 3360-3368. doi: 10.4049/jimmunol. 1700893. Epub 2017 Oct. 4.
O'Donnell T J, Rubinsteyn A, Bonsack M, Riemer A B, Laserson U, and Hammerbacher J. MHCflurry: Open-Source Class I MHC Binding Affinity Prediction. Cell Syst. 2018 Jul. 25; 7(1): 129-132.e4. doi: 10.1016/j.cels.2018.05.014. Epub 2018 Jun. 27.
Cameron B J, Gerry A B, Dukes J, Harper J V, Kannan V, Bianchi F C, Grand F, Brewer J E, Gupta M, Plesa G, et al. Identification of A Titin-Derived HLA-A1-Presented Peptide as a Cross-Reactive Target for Engineered Mage A3-directed T Cells. Sci Transl Med. 2013; 5 (197): 197ra103. doi: 10.1126/scitranslmed.3006034.
Linette G P, Stadtmauer E A, Maus M V, Rapoport A P, Levine B L, Emery L, Litzky L, Bagg A, Carreno B M, Cimino P J, et al. Cardiovascular Toxicity and Titin Cross-Reactivity of Affinity-Enhanced T Cells in Myeloma and Melanoma. Blood. 2013; 122 (6): 863-71. doi: 10.1182/blood-2013-03-490565.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-85. (canceled)

86. An in vitro method of assessing off-target effects of one or more antigen-recognition molecules that bind a target peptide presented in a complex with a major histocompatibility complex (MHC) molecule (MHC-target peptide complex), comprising:
a) contacting each of the one or more antigen-recognition molecules with one or more off-target peptides, wherein each of said one or more off-target peptides is presented in a complex with the same type of MHC molecule (MHC-off-target peptide complex); and
b) determining the level of binding of each of the one or more antigen-recognition molecules to each of the one or more MHC-off-target peptide complexes, wherein:
(i) the target peptide is a MAGEA4₂₃₀₋₂₃₉ peptide comprising the amino acid sequence GVYDGREHTV (SEQ ID NO: 1) or a pharmaceutically acceptable salt thereof, and the one or more off-target peptides comprise an amino acid sequence selected from SEQ ID NOs: 2-7, 9, 11-28, 73-74, or a pharmaceutically acceptable salt thereof, or
(ii) the target peptide is a MAGEA4₂₈₆₋₂₉₄ peptide comprising the amino acid sequence KVLEHVVRV (SEQ ID NO: 48) or a pharmaceutically acceptable salt thereof, and the one or more off-target peptides comprise an amino acid sequence selected from SEQ ID NOs: 49-72, or a pharmaceutically acceptable salt thereof, or
(iii) the target peptide is a MAGEA3₁₆₈₋₁₇₆ peptide comprising the amino acid sequence EVDPIGHLY (SEQ ID NO: 29) or a pharmaceutically acceptable salt thereof, and the one or more off-target peptides comprise an amino acid sequence selected from SEQ ID NOs: 30-47, or a pharmaceutically acceptable salt thereof.

87. The method of claim 86, wherein at least one of the one or more antigen-recognition molecules detectably binds to at least one of the one or more MHC-off-target peptide complexes.

88. The method of claim 86, further comprising determining the level of binding of the one or more antigen-recognition molecules to the MHC-target peptide complex.

89. The method of claim 86, wherein the one or more antigen-recognition molecules comprises a plurality of antigen-recognition molecules.

90. The method of claim 89, further comprising:
c) selecting one or more antigen-recognition molecules based at least in part on the number of MHC-off-target peptide complexes detectably bound by each of the antigen-recognition molecules; and
d) repeating steps (a) and (b) using the selected one or more antigen-recognition molecules.

91. The method of claim 90, wherein the one or more selected antigen-recognition molecules do not detectably bind to any of the MHC-off-target peptide complexes.

92. The method of claim 89, wherein the plurality of antigen-recognition molecules is in a library.

93. The method of claim 92, wherein the library is a phage display library or a yeast library.

94. The method of claim 86, wherein the one or more MHC-off-target peptide complexes are each immobilized on a solid support.

95. The method of claim 86, wherein the one or more MHC-off-target peptide complexes are each presented on a cell surface.

96. The method of claim 86, wherein the level of binding is determined by detecting the amount of each of the one or more antigen-recognition molecules bound to each of the one or more MHC-off-target peptide complex.

97. The method of claim 86, wherein the method is performed in a high-throughput format.

98. The method of claim 97, wherein the method is performed in a multi-well plate.

99. The method of claim 86, wherein the MHC molecule of the MHC-target peptide complex is an HLA-A molecule and the MHC molecule of each of the one or more MHC-off-target peptide complexes is an HLA-A molecule.

100. The method of claim 99, wherein:
(i) the target peptide is the MAGEA4₂₃₀₋₂₃₉ peptide or a pharmaceutically acceptable salt thereof and the MHC molecule of the MHC-target peptide complex is an HLA-A2 molecule and the MHC molecule of each of the one or more MHC-off-target peptide complexes is an HLA-A2 molecule, or
(ii) the target peptide is the MAGEA4₂₈₆₋₂₉₄ peptide or a pharmaceutically acceptable salt thereof and the MHC molecule of the MHC-target peptide complex is an HLA-A2 molecule and the MHC molecule of each of the one or more MHC-off-target peptide complexes is an HLA-A2 molecule, or
(iii) the target peptide is the MAGEA3₁₆₈₋₁₇₆ peptide or a pharmaceutically acceptable salt thereof and the MHC molecule of the MHC-target peptide complex is an HLA-A1 molecule and the MHC molecule of each of the one or more MHC-off-target peptide complexes is an HLA-A1 molecule.

101. The method of claim 86, wherein the target peptide is the MAGEA4₂₃₀₋₂₃₉ peptide or a pharmaceutically acceptable salt thereof and the one or more off-target peptides comprise the amino acid sequence of SEQ ID NO: 3, or a pharmaceutically acceptable salt thereof.

102. A library comprising:
a) two or more off-target peptides comprising amino acid sequences selected from SEQ ID NOs: 2-7, 9, 11-28, 73-74 or a pharmaceutically acceptable salt thereof; and/or
b) two or more off-target peptides comprising amino acid sequences selected from SEQ ID NOs: 49-72 or a pharmaceutically acceptable salt thereof; and/or
c) two or more off-target peptides comprising amino acid sequences selected from SEQ ID NOs: 30-47 or a pharmaceutically acceptable salt thereof.

103. A library comprising two or more off-target peptides or pharmaceutically acceptable salts thereof, wherein said two or more peptides are identified using a method comprising:
a) predicting all amino acid positions within a target peptide presented in a complex with a major histocompatibility complex (MHC) molecule (MHC-target peptide complex) that are available to interact with antigen-recognition molecules;
b) identifying within a total pool of predicted or detected peptides of suitable length similar peptides that include at least two amino acids that (i) are located at positions corresponding to the amino acid positions within the target peptide predicted in step (a) and (ii) are identical to amino acids of the target peptide at the amino acid positions within the target peptide predicted in step (a);
c) determining binding affinity of each of the similar peptide(s) identified in step (b) to the MHC molecule; and
d) identifying off-target peptide(s) based at least in part on identifying similar peptide(s) having a calculated binding affinity to the MHC molecule stronger than a first threshold value.

104. The library of claim 103, wherein step (a) comprises analyzing one or more experimental structures of the MHC-target peptide complex with a specific antigen-recognition molecule.

105. The library of claim 103, wherein step (a) comprises:
(i) determining binding affinity of the target peptide to the MHC molecule,
(ii) generating a plurality of mutated peptides each associated with a mutation at a respective amino acid position of the target peptide,
(iii) determining binding affinity of each mutated peptide of the plurality of mutated peptides to the MHC molecule, and
(iv) predicting the amino acid position(s) available for interacting with antigen-recognition molecules based at least in part on a comparison of the binding affinity for each mutated peptide to the binding affinity of the target peptide.
